SEQUENCE LISTING (18 sequences)

SEQ ID NO: 1 - protein, 219 aa, synthetic construct
  REGION 1..219: note = DuetMab PD-1 (LO115) Light Chain
QIVLTQSPATLSLSPGERATLSCSASSKHTNLYWSRHMYWYQQKPGQAPRLLIYLTSNRATGIPARFSGSGSGTDFTLTISSLEPEDFAVYYCQQWSSNPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 452 aa, synthetic construct
  REGION 1..452: note = DuetMab PD-1 (LO115) Heavy Chain
EVQLVESGGGLVQPGGSLRLSCAASGFTFSDYGMHWVRQAPGKGLEWVAYISSGSYTIYSADSVKGRFTISRDNAKNSLYLQMNSLRAEDTAVYYCARRAPNSFYEYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 3 - protein, 213 aa, synthetic construct
  REGION 1..213: note = DuetMab CTLA-4 Light Chain
DIQMTQSPSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKGQPKAAPSVTLFPPCSEELQANKATLVCLISDFYPGAVTVAWKADSSPVKAGVETTTPSKQSNNKYAASSYLSLTPEQWKSHRSYSCQVTHEGSTVEKTVAPTEVS

SEQ ID NO: 4 - protein, 455 aa, synthetic construct
  REGION 1..455: note = DuetMab CTLA-4 Heavy Chain
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSVDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 18 aa, synthetic construct
  REGION 1..18: note = PD-1 (LO115) LC - CDR1
SASSKHTNLYWSRHMYWY

SEQ ID NO: 6 - protein, 6 aa, synthetic construct
  REGION 1..6: note = PD-1 (LO115) LC - CDR2
TSNRAT

SEQ ID NO: 7 - protein, 7 aa, synthetic construct
  REGION 1..7: note = PD-1 (LO115) LC - CDR3
QQWSSNP

SEQ ID NO: 8 - protein, 10 aa, synthetic construct
  REGION 1..10: note = PD-1 (LO115) HC - CDR1
GFTFSDYGMH

SEQ ID NO: 9 - protein, 17 aa, synthetic construct
  REGION 1..17: note = PD-1 (LO115) HC - CDR2
YISSGSYTIYSADSVKG

SEQ ID NO: 10 - protein, 13 aa, synthetic construct
  REGION 1..13: note = PD-1 (LO115) HC - CDR3
RAPNSFYEYYFDY

SEQ ID NO: 11 - protein, 11 aa, synthetic construct
  REGION 1..11: note = CTLA-4 LC - CDR1
RASQSINSYLD

SEQ ID NO: 12 - protein, 7 aa, synthetic construct
  REGION 1..7: note = CTLA-4 LC - CDR2
AASSLQS

SEQ ID NO: 13 - protein, 7 aa, synthetic construct
  REGION 1..7: note = CTLA-4 LC - CDR3
QQYYSTP

SEQ ID NO: 14 - protein, 10 aa, synthetic construct
  REGION 1..10: note = CTLA-4 HC - CDR1
GFTFSSYGMH

SEQ ID NO: 15 - protein, 17 aa, synthetic construct
  REGION 1..17: note = CTLA-4 HC - CDR2
VIWYDGSNKYYADSVKG

SEQ ID NO: 16 - protein, 16 aa, synthetic construct
  REGION 1..16: note = CTLA-4 HC - CDR3
DPRGATLYYYYYGMDV

SEQ ID NO: 17 - protein, 288 aa, synthetic construct
  REGION 1..288: note = PD-1 Amino Acid Sequence
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 18 - protein, 223 aa, synthetic construct
  REGION 1..223: note = CTLA-4 Amino Acid Sequence
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

(19) United States
(12) Patent Application Publication
(10) Pub. No.: US 2023/0322928 A1
(43) Pub. Date: Oct. 12, 2023

(54) TREATMENT METHODS USING CTLA-4 AND PD-1 BISPECIFIC ANTIBODIES

(71) Applicant: MedImmune, LLC, Gaithersburg, US (US)

(72) Inventors: Daniel J. FREEMAN, Gaithersburg, MD (US); Xuyang SONG, Gaithersburg, MD (US); Shelby GAINER, Gaithersburg, MD (US); Deepa SUBRAMANIAM, Gaithersburg, MD (US); Ikbel ACHOUR, Gaithersburg, MD (US); Charles FERTE, Gaithersburg, MD (US)

(21) Appl. No.: 18/178,687

(22) Filed: Mar. 6, 2023

Related U.S. Application Data
(60) Provisional application No. 63/374,815, filed on Sep. 7, 2022.
(60) Provisional application No. 63/347,748, filed on Jun. 1, 2022.
(60) Provisional application No. 63/317,200, filed on Mar. 7, 2022.

(51) Int. Cl. C07K 16/28; A61P 35/00; A61K 31/4439; A61K 31/47

(52) U.S. Cl. CPC C07K 16/2818; A61P 35/00; A61K 31/4439; A61K 31/47; A61K 2039/545

(57) ABSTRACT

The present disclosure provides methods of administering bispecific antibodies and antigen-binding fragments thereof that specifically bind to human Programmed cell-death-1 (PD-1) and Cytotoxic T-lymphocyte-associated antigen-4 (CTLA-4) to a subject in need thereof, for example, a subject with cancer.

### CROSS-REFERENCE TO RELATED APPLICATIONS

This application claims priority from U.S. provisional application Nos. 63/317,200, filed Mar. 7, 2022; 63/347,748, filed Jun. 1, 2022; and 63/374,815, filed Sep. 7, 2022, each of which are incorporated herein by reference.

### REFERENCE TO SEQUENCE LISTING

The content of the electronically submitted sequence listing (Name: PDCT-200-WO-PCT.xml, Size: 26.6 KB, and Date of Creation: Mar. 3, 2023) submitted in this application is incorporated herein by reference in its entirety.

### 1. FIELD

The present disclosure relates generally to methods of using bispecific antibodies and antigen-binding fragments thereof that specifically bind to human Programmed cell-death-1 (PD-1) and Cytotoxic T-lymphocyte-associated Antigen-4 (CTLA-4) for the treatment of cancers, e.g., renal cell carcinomas and non-small cell lung cancers.

### 2. BACKGROUND

Cancer continues to be a major global health burden. Despite progress in the treatment of cancer, there continues to be an unmet medical need for more effective and less toxic therapies, especially for those patients with advanced disease or cancers that are resistant to existing therapeutics.

The role of the immune system, in particular T cell-mediated cytotoxicity, in tumor control is well recognized. There is mounting evidence that T cells control tumor growth and survival in cancer patients, both in early and late stages of the disease. However, tumor-specific T-cell responses are difficult to mount and sustain in cancer patients. The continuing advancement and successes of cancer immunotherapies, which stimulate or enhance innate immune responses against cancer, make such therapeutics an attractive treatment option when compared to therapies that utilize non-specific chemotherapeutics and/or radiation.

A number of molecular targets have been identified for their potential utility as immuno-oncology (IO) therapeutics against cancer. Some molecular targets that are being investigated for their therapeutic potential in the area of immuno-oncology therapy include cytotoxic T lymphocyte antigen-4 (CTLA-4 or CD152), programmed death ligand 1 (PD-L1 or B7-H1 or CD274), Programmed Death-1 (PD-1), OX40 (CD134 or TNFRSF4) and T-cell inhibitory receptor T-cell immunoglobulin and mucin-domain containing-3 (TIM3). While some of these targets have been successfully exploited therapeutically (e.g., PD-1 and CTLA-4), many patients have been unresponsive to the therapeutics that have been developed. And, while a therapeutic regimen that includes higher doses and/or a combination of immunotherapies may be considered, such therapies may be associated with increased risk of side effects, which tend to increase with higher doses and cumulative exposure, and appear to be additive when used with combination immunotherapies. Some common side effects include hypophysitis, thyroiditis, adrenal insufficiency, enterocolitis, dermatitis, pneumonitis, hepatitis, pancreatitis, motor and sensory neuropathies, and arthritis. Furthermore, as immunotherapeutics are typically associated with high costs, a therapy that includes a combination of immunotherapeutics can be cost-prohibitive to patients.

As such, there remains a need to continue to identify candidate targets for IO therapeutics, develop new therapeutics to the existing targets, and to develop therapeutic strategies that avoid disadvantages of immunotherapies that are currently in use, including the lack of patient response and the increased risk of side effects involved with combination treatment. IO therapeutics (e.g., binding proteins) that are bispecific for a combination of target molecules, particularly those that exhibit greater binding affinity for the target molecules when compared to the binding affinity for a combination of individual monospecific binding proteins, represent a class of particularly desirable molecules for therapeutic potential.

### 3. SUMMARY

In some aspects, disclosed herein is a method of treating renal cell carcinoma (RCC) in a subject, the method comprising administering to the subject about 250 mg to about 1500 mg of a bispecific antibody or antigen-binding fragment thereof that specifically binds to Programmed cell-death-1 (PD-1) and Cytotoxic T-lymphocyte-associated antigen-4 (CTLA-4). In some aspects, disclosed herein is a method of treating renal cell carcinoma (RCC) in a subject, the method comprising administering to the subject about 2.25 mg to about 2500 mg of a bispecific antibody or antigen-binding fragment thereof that specifically binds to Programmed cell-death-1 (PD-1) and Cytotoxic T-lymphocyte-associated antigen-4 (CTLA-4).

In some aspects, the methods comprise administering about 500 mg or about 750 mg of the bispecific antibody or antigen-binding fragment thereof.

In some aspects, the methods further comprise administering one or more tyrosine kinase inhibitors.

In some aspects, the methods further comprise administering the tyrosine kinase inhibitor axitinib or lenvatinib.

In some aspects, the axitinib is orally administered at a dose of 5 mg twice daily, from day −7 to day −1, prior to administration of the bispecific antibody or antigen-binding fragment thereof.

In some aspects, the bispecific antibody or antigen-binding fragment thereof is administered on day 1.

In some aspects, the bispecific antibody or antigen-binding fragment thereof is administered at a dose of 500 mg or 750 mg once per treatment cycle.

In some aspects, the treatment cycle is three weeks.

In some aspects, the methods further comprise maintenance dosing of the bispecific antibody or antigen-binding fragment thereof and/or the one or more doses of the chemotherapeutic agents.

In some aspects, disclosed herein is a method of treating non-small cell lung cancer (NSCLC) in a subject, the method comprising administering to the subject about 250 mg to about 1500 mg of a bispecific antibody or antigen-binding fragment thereof that specifically binds to Programmed cell-death-1 (PD-1) and Cytotoxic T-lymphocyte-associated antigen-4 (CTLA-4). In some aspects, disclosed herein is a method of treating non-small cell lung cancer (NSCLC) in a subject, the method comprising administering to the subject about 2.25 mg to about 2500 mg of a bispecific antibody or antigen-binding fragment thereof that specifically binds to Programmed cell-death-1 (PD-1) and Cytotoxic T-lymphocyte-associated antigen-4 (CTLA-4).

In some aspects, disclosed herein is a method of inhibiting growth of a non-small cell lung tumor in a subject, the method comprising administering to the subject about 250 mg to about 1500 mg of a bispecific antibody or antigen-binding fragment thereof that specifically binds Programmed cell-death-1 (PD-1) and Cytotoxic T-lymphocyte-associated antigen-4 (CTLA-4). In some aspects, disclosed herein is a method of inhibiting growth of a non-small cell lung tumor in a subject, the method comprising administering to the subject about 2.25 mg to about 2500 mg of a bispecific antibody or antigen-binding fragment thereof that specifically binds Programmed cell-death-1 (PD-1) and Cytotoxic T-lymphocyte-associated antigen-4 (CTLA-4).

In some aspects, the method comprises administering about 500 mg or about 750 mg of the bispecific antibody or antigen-binding fragment thereof.

In some aspects, the bispecific antibody or an antigen-binding fragment thereof is administered once per treatment cycle.

In some aspects, the treatment cycle is three weeks.

In some aspects, the method comprises administering one or more chemotherapeutic agents.

In some aspects, the NSCLC or non-small cell lung tumor is a non-squamous cell lung carcinoma.

In some aspects, the NSCLC or non-small cell lung tumor is a squamous cell lung carcinoma.

In some aspects, the one or more chemotherapeutic agents selected from the group consisting of carboplatin, pemetrexed, and a combination thereof.

In some aspects, the one or more chemotherapeutic agents are carboplatin and pemetrexed. In some aspects, the NSCLC or non-small cell lung tumor is a non-squamous cell lung carcinoma and the one or more chemotherapeutic agents are carboplatin and pemetrexed.

In some aspects, the bispecific antibody or antigen-binding fragment thereof is administered at a dose of 500 mg or 750 mg every three weeks and the carboplatin is administered at a dose of AUC 6 mg/mL·min or AUC 5 mg/mL·min every three weeks.

In some aspects, the bispecific antibody or antigen-binding fragment thereof is administered at a dose of about 2000 mg, the carboplatin is administered at a dose of AUC 5 mg/mL·min and the pemetrexed is administered at a dose of 500 mg/m².

In some aspects, the bispecific antibody or antigen-binding fragment thereof is administered at a dose of about 1500 mg, the carboplatin is administered at a dose of AUC 5 mg/mL·min and the pemetrexed is administered at a dose of 500 mg/m², every three weeks, wherein the carboplatin is administered for 4 doses, followed by maintenance dosing of the bispecific antibody or antigen-binding fragment thereof every three weeks and pemetrexed every three weeks.

In some aspects, the bispecific antibody or antigen-binding fragment thereof is administered at a dose of about 500 mg or about 750 mg, the carboplatin is administered at a dose of AUC 5 mg/mL·min and the pemetrexed is administered at a dose of 500 mg/m², every three weeks, wherein the carboplatin is administered for 4 doses, followed by maintenance dosing of the bispecific antibody or antigen-binding fragment thereof every three weeks and maintenance dosing of the pemetrexed every three weeks.

In some aspects, the one or more chemotherapeutic agents is selected from the group consisting of carboplatin, paclitaxel, Nab-paclitaxel, and a combination thereof.

In some aspects, the one or more chemotherapeutic agents are carboplatin and paclitaxel.

In some aspects, the one or more chemotherapeutic agents are carboplatin and Nab-paclitaxel. In some aspects, the NSCLC or non-small cell lung tumor is a squamous cell lung carcinoma and the one or more chemotherapeutic agents are carboplatin and Nab-paclitaxel.

In some aspects, the bispecific antibody or antigen-binding fragment thereof is administered at a dose of 500 mg or 750 mg every three weeks, the carboplatin is administered at a dose of AUC 6 mg/mL·min every three week, and the paclitaxel is administered at a dose of 200 mg/m² every three weeks. In some aspects, the carboplatin and the paclitaxel are administered for 4 doses.

In some aspects, the bispecific antibody or antigen-binding fragment thereof is administered at a dose of 500 mg or 750 mg every three weeks, the carboplatin is administered at a dose of AUC 6 mg/mL·min every three week, and the nab-paclitaxel is administered at a dose of 100 mg/m² body surface area (BSA) on Days 1, 8, and 15 of each 3-week cycle. In some aspects, the carboplatin and the nab-paclitaxel are administered for 4 doses.

In some aspects, the method further comprises maintenance dosing of the bispecific antibody or antigen-binding fragment thereof and/or the one or more chemotherapeutic agents.

In some aspects, the subject is human.

In some aspects, the subject has an advancing solid tumor.

In some aspects, the administration results in the disappearance and/or decrease in sum of a target lesion.

In some aspects, the disappearance and/or decrease in sum of a target lesion is determined by assaying a tumor biopsy sample.

In some aspects, the sample is a fresh or formalin-fixed paraffin embedded (FFPE) sample.

In some aspects, the sample is assayed by RT-PCR, in situ hybridization, RNase protection, RT-PCR-based assay, immunohistochemistry (IHC), enzyme linked immuosorbent assay, in vivo imaging, or flow cytometry.

In some aspects, the bispecific antibody binds to cynomolgus monkey PD-1 and CTLA-4.

In some aspects, the bispecific antibody or antigen binding fragment thereof binds to human PD-1 and CTLA-4.

In some aspects, the bispecific antibody or antigen-binding fragment thereof is a humanized bispecific antibody or antigen-binding fragment thereof.

In some aspects, the bispecific antibody or antigen-binding fragment thereof is monovalent.

In some aspects, the bispecific antibody or antigen-binding fragment thereof is a DuetMab.

In some aspects, the bispecific antibody or antigen-binding fragment comprises an IgG heavy chain constant region.

In some aspects, the constant region includes mutations at L234F, L235E and P331S.

In some aspects, the constant region comprises a knob mutation and a hole mutation, optionally wherein the knob mutation is in a heavy chain comprising a variable region that binds to CTLA-4 and the hole mutation is in a heavy chain comprising a variable region that binds to PD-1.

In some aspects, the IgG heavy chain constant region is an IgG1 heavy chain constant region.

In some aspects, the bispecific antibody or antigen-binding fragment thereof comprises the anti-PD-1 and anti-CTLA-4 heavy chain variable region (VH) CDR1, VH CDR2, VH CDR3, light chain variable region (VL) CDR1, VL CDR2, and VL CDR3 of sequences of MEDI5752.

In some aspects, the bispecific antibody or antigen-binding fragment thereof comprises: (a) a VH CDR1 comprising the amino acid sequence of SEQ ID NO:8, a VH CDR2 comprising the amino acid sequence of SEQ ID NO:9, a VH CDR3 comprising the amino acid sequence of SEQ ID NO:10, a VL CDR1 comprising the amino acid sequence of SEQ ID NO:5, a VL CDR2 comprising the amino acid sequence of SEQ ID NO:6, and a VL CDR3 comprising the amino acid sequence of SEQ ID NO:7; and (b) a VH CDR1 comprising the amino acid sequence of SEQ ID NO:14, a VH CDR2 comprising the amino acid sequence of SEQ ID NO:15, a VH CDR3 comprising the amino acid sequence of SEQ ID NO:16, a VL CDR1 comprising the amino acid sequence of SEQ ID NO:11, a VL CDR2 comprising the amino acid sequence of SEQ ID NO:12, and a VL CDR3 comprising the amino acid sequence of SEQ ID NO:13.

In some aspects, the bispecific antibody or antigen-binding fragment thereof comprises: (a) a heavy chain comprising the amino acid sequence set forth in SEQ ID NO:2 and a light chain comprising the amino acid sequence set forth in SEQ ID NO:1; and (b) a heavy chain comprising the amino acid sequence set forth in SEQ ID NO:4 and a light chain comprising the amino acid sequence set forth in SEQ ID NO:3.

In some aspects, the bispecific antibody or antigen-binding fragment thereof is a full-length antibody.

In some aspects, the bispecific antibody is MEDI5752.

In some aspects, the bispecific antibody or antigen-binding fragment thereof is an antigen binding fragment.

In some aspects, the antigen binding fragment comprises a Fab, Fab′, F(ab′)2, single chain Fv (scFv), disulfide linked Fv, V-NAR domain, IgNar, intrabody, bispecific intrabody, IgGΔCH2, minibody, F(ab′)3, tetrabody, triabody, diabody, single-domain antibody, DVD-Ig, Fcab, mAb2, (scFv)2, or scFv-Fc.

In some aspects, provided herein is a method of treating advanced renal cell carcinoma in a subject, the method comprising administering to the subject about 750 mg of a bispecific antibody that specifically binds to PD-1 and CTLA-4, and comprises: (a) a heavy chain comprising the amino acid sequence set forth in SEQ ID NO:2 and a light chain comprising the amino acid sequence set forth in SEQ ID NO:1; and (b) a heavy chain comprising the amino acid sequence set forth in SEQ ID NO:4 and a light chain comprising the amino acid sequence set forth in SEQ ID NO:3, every three weeks.

In some aspects, provided herein is a method of treating advanced renal cell carcinoma in a subject, the method comprising administering to the subject about 500 mg of a bispecific antibody that specifically binds to PD-1 and CTLA-4, and comprises: (a) a heavy chain comprising the amino acid sequence set forth in SEQ ID NO:2 and a light chain comprising the amino acid sequence set forth in SEQ ID NO:1; and (b) a heavy chain comprising the amino acid sequence set forth in SEQ ID NO:4 and a light chain comprising the amino acid sequence set forth in SEQ ID NO:3, every three weeks.

In some aspects, provided herein is a method of treating non-small cell lung cancer in a subject, the method comprising administering to the subject: (1) about 750 mg or about 500 mg of a bispecific antibody that specifically binds to PD-1 and CTLA-4, and comprises: (a) a heavy chain comprising the amino acid sequence set forth in SEQ ID NO:2 and a light chain comprising the amino acid sequence set forth in SEQ ID NO:1; and (b) a heavy chain comprising the amino acid sequence set forth in SEQ ID NO:4 and a light chain comprising the amino acid sequence set forth in SEQ ID NO:3 every three weeks; (2) carboplatin at a dose of AUC 5 mg/mL·min every three weeks for 4 cycles; and (3) pemetrexed at a dose of 500 mg/m2 every three weeks for cycles, followed by maintenance dosing of the bispecific antibody and pemetrexed.

In some aspects, provided herein is a method of treating non-small cell lung cancer in a subject, the method comprising administering to the subject: (1) about 750 mg or about 500 mg of a bispecific antibody that specifically binds to PD-1 and CTLA-4, and comprises: (a) a heavy chain comprising the amino acid sequence set forth in SEQ ID NO:2 and a light chain comprising the amino acid sequence set forth in SEQ ID NO:1; and (b) a heavy chain comprising the amino acid sequence set forth in SEQ ID NO:4 and a light chain comprising the amino acid sequence set forth in SEQ ID NO:3 every three weeks; and (2) carboplatin at a dose of AUC 6 mg/mL·min every three weeks; and (3) paclitaxel at a dose of 200 mg/m2 every three weeks, or (4) Nab-paclitaxel at a dose of 100 mg/m2 body surface area on days 1, 8, and 15 of every three week cycle for 4 cycles, and (5) maintenance dosing of the bispecific antibody.

In some aspects, the non-small cell lung cancer and/or the renal cell carcinoma comprises about ≥50% of the tumor cells expressing PD-L1. In some aspects, the non-small cell lung cancer and/or the renal cell carcinoma comprises about 1-49% of the tumor cells expressing PD-L1. In some aspects, the non-small cell lung cancer and/or the renal cell carcinoma comprises about <1% of the tumor cells expressing PD-L1. In some aspects, the non-small cell lung cancer and/or the renal cell carcinoma comprises about 0% of the tumor cells expressing PD-L1.

In some aspects, the present disclosure also provides a method of expanding T cells in a subject in need thereof, comprising administering a bispecific antibody or antigen-binding fragment thereof that specifically binds to Programmed cell-death-1 (PD-1) and Cytotoxic T-lymphocyte-associated antigen-4 (CTLA-4), the antibdy comprising: (a) a VH CDR1 comprising the amino acid sequence of SEQ ID NO:8, a VH CDR2 comprising the amino acid sequence of SEQ ID NO:9, a VH CDR3 comprising the amino acid sequence of SEQ ID NO:10, a VL CDR1 comprising the amino acid sequence of SEQ ID NO:5, a VL CDR2 comprising the amino acid sequence of SEQ ID NO:6, and a VL CDR3 comprising the amino acid sequence of SEQ ID NO:7; and (b) a VH CDR1 comprising the amino acid sequence of SEQ ID NO:14, a VH CDR2 comprising the amino acid sequence of SEQ ID NO:15, a VH CDR3 comprising the amino acid sequence of SEQ ID NO:16, a VL CDR1 comprising the amino acid sequence of SEQ ID NO:11, a VL CDR2 comprising the amino acid sequence of SEQ ID NO:12, and a VL CDR3 comprising the amino acid sequence of SEQ ID NO:13. In some aspects, the bispecific antibody comprises: (a) a heavy chain comprising the amino acid sequence set forth in SEQ ID NO:2 and a light chain comprising the amino acid sequence set forth in SEQ ID NO:1; and (b) a heavy chain comprising the amino acid sequence set forth in SEQ ID NO:4 and a light chain comprising the amino acid sequence set forth in SEQ ID NO:3. In some aspects, the bispecific antibody is administered at a dose of about 225 mg to about 1,500 mg. In some aspects, the bispecific antibody is administered at a dose of about 750 mg Q3W. In some aspects, the bispecific antibody is administered every three weeks. In some aspects, the subject has non-small cell lung cancer or renal cell carcinoma. In some aspects, the proportion of newly expanded T cell clones is about 50%, about 60%, about 70%, or about 75% higher compared to the number of T cell clones prior to administration. In some aspects, the bispecific antibody is MEDI5752.

### 4. BRIEF DESCRIPTION OF THE FIGURES

FIGS. 1A-1D is a study flow diagram for the first-in-human dose escalation (FIG. 1A) and dose expansion (FIGS. 1B-1D) study of MEDI5752.

FIG. 2 shows the predicted MEDI5752 concentration-time profiles following IV infusion of MEDI5752 Q3W with projected EC₂₀, EC₅₀, and EC₉₀ for PD-1.

FIG. 3 shows the predicted MEDI5752 concentration-time profiles following IV infusion of MEDI5752 Q3W with projected EC₂₀, and EC₉₀ for CTLA-4 and EC₂₀, and EC₉₀ PD-1.

FIG. 4 shows mean MEDI5752 pharmacokinetic profiles following IV infusion of MEDI5752 Q3W.

FIG. 5 shows Exposure (C_{trough}) v. CD4 T cell proliferation following MEDI5752 treatment.

FIG. 6 shows percent free PD1 on CD4 T cells (PD1 Receptor occupancy measured longitudinally by flow cytometry following MEDI5752.

FIGS. 7A-7C show peripheral blood T cell proliferation (CD4+ Ki67+) (FIG. 7A) and (FIG. 7C) and T cell activation (ICOS expression on CD4 T cells) (FIG. 7B) as measured by flow cytometry following MEDI5752 as monotherapy or in combination with chemotherapy

FIGS. 8A-8C show total of expanded T cell clones (FIG. 8A) and proportion of newly expanded T cell clones (FIGS. 8B and 8C) as measured by T cell receptor sequencing (TCRseq) following MEDI5752 as monotherapy or in combination with chemotherapy.

FIG. 9 shows the median duration of response for MEDI5752 monotherapy at various doses.

FIG. 10 shows the objective responses of MEDI5752 monotherapy in diverse IO-naïve tumors across a range of MEDI5752 doses.

FIG. 11 shows that the efficacy of 750 mg was similar to 1500 mg MEDI5752 monotherapy.

FIG. 12 shows responses for the first-line Renal Cell Carcinoma cohort with treatment of MEDI5752 at 1,500 mg Q3W (top), 750 mg Q3W (middle), and 500 mg Q3W (bottom).

FIG. 13 shows the change from baseline (%) for the first-line RCC cohort for 750 mg and 1500 mg MEDI5752 monotherapy and median duration of response and median PFS for those with first-line RCC treated at 1500 mg.

FIG. 14 shows Progression Free Survival (PFS) in patients treated with MEDI5752 1,500 mg and chemotherapy compared to patients treated with pembolizumab and chemotherapy. The MEDI5752 treatment had 20 subjects (11 events) with a median PFS of 15.1 months. The pembrolizumab treatment had 21 subjects (16 events) with a median PFS of 8.9 months.

FIG. 15A shows a waterfall plot of efficacy of treatment with MEDI5752 750 mg and chemotherapy in PD-L1 <1%, PD-L1 1-49%, PD-L1 >50%, and PD-L1 not evaluable NSCLC patients. FIG. 15B shows a waterfall plot of efficacy of treatment of non-squamous NSCLC-Cohort 2, with subjects treated with 500 mg MEDI5752+carboplatin/pemetrexed and 750 mg MEDI5752+carboplatin/pemetrexed. FIG. 15C shows a waterfall plot of the squamous NSCLC expansion cohort 2 patients treated with 750 mg MEDI5752+carboplatin/pemetrexed.

### 5. DETAILED DESCRIPTION

In order that the present disclosure may be more readily understood, certain terms are first defined. As used in this application, except as otherwise expressly provided herein, each of the following terms shall have the meaning set forth below. Additional definitions are set forth throughout the application.

### 5.1 Definitions

In order that the present disclosure may be more readily understood, certain terms are first defined. As used in this application, except as otherwise expressly provided herein, each of the following terms shall have the meaning set forth below. Additional definitions are set forth throughout the application.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, chimeric antibodies, humanized antibodies, human antibodies, fusion proteins comprising an antibody, and any other modified immunoglobulin molecule so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

Where not expressly stated, and unless the context indicates otherwise, the term “antibody” includes monospecific, bispecific, or multi-specific antibodies, as well as a single chain antibody. In some aspects, the antibody is a bispecific antibody. The term “bispecific antibodies” refers to antibodies that bind to two different epitopes. The epitopes can be on the same target antigen or can be on different target antigens.

The term “antibody fragment” refers to a portion of an intact antibody. An “antigen-binding fragment,” “antigen-binding domain,” or “antigen-binding region,” refers to a portion of an intact antibody that binds to an antigen. In the context of a bispecific antibody, an “antigen-binding fragment binds two antigens. An antigen-binding fragment can contain an antigen recognition site of an intact antibody (e.g., complementarity determining regions (CDRs) sufficient to specifically bind antigen). Examples of antigen-binding fragments of antibodies include, but are not limited to Fab, Fab′, F(ab′)2, and Fv fragments, linear antibodies, and single chain antibodies. An antigen-binding fragment of an antibody can be derived from any animal species, such as rodents (e.g., mouse, rat, or hamster) and humans or can be artificially produced.

A “monoclonal” antibody or antigen-binding fragment thereof refers to a homogeneous antibody or antigen-binding fragment population involved in the highly specific binding of a single antigenic determinant, or epitope. This is in contrast to polyclonal antibodies that typically include different antibodies directed against different antigenic determinants. The term “monoclonal” antibody or antigen-binding fragment thereof encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal” antibody or antigen-binding fragment thereof refers to such antibodies and antigen-binding fragments thereof made in any number of manners including but not limited to by hybridoma, phage selection, recombinant expression, and transgenic animals.

In some aspects, the antibodies or antigen binding fragments thereof disclosed herein are multivalent molecules. The term “valent” as used within the current application denotes the presence of a specified number of binding sites in an antibody molecule. A natural antibody for example or a full length antibody according to the invention has two binding sites and is “bivalent.” The term “tetravalent,” denotes the presence of four binding sites in an antigen binding protein. The term “trivalent” denotes the presence of three binding sites in an antibody molecule. The term “bispecific, tetravalent,” as used herein denotes an antigen binding protein according to the invention that has four antigen-binding sites of which at least one binds to a first antigen and at least one binds to a second antigen or another epitope of the antigen.

As used herein, the terms “variable region” or “variable domain” are used interchangeably and are common in the art. The variable region typically refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that CDRs of the light and heavy chains are primarily responsible for the interaction and specificity of the antibody with antigen. In some aspects of the present disclosure, the variable region is a human variable region. In some aspects of the present disclosure, the variable region comprises rodent or murine CDRs and human framework regions (FRs). In particular aspects of the present disclosure, the variable region is a primate (e.g., non-human primate) variable region. In some aspects of the present disclosure, the variable region comprises rodent or murine CDRs and primate (e.g., non-human primate) framework regions (FRs).

The terms “VL” and “VL domain” are used interchangeably to refer to the light chain variable region of an antibody.

The terms “VH” and “VH domain” are used interchangeably to refer to the heavy chain variable region of an antibody.

The term “Kabat numbering” and like terms are recognized in the art and refer to a system of numbering amino acid residues in the heavy and light chain variable regions of an antibody or an antigen-binding fragment thereof. In some aspects, CDRs can be determined according to the Kabat numbering system (see, e.g., Kabat EA & Wu TT (1971) Ann NY Acad Sci 190: 382-391 and Kabat EA et al., (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3). In some aspects of the present disclosure, the CDRs of the antibodies described herein have been determined according to the Kabat numbering scheme.

Chothia refers instead to the location of the structural loops (Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software.

| Loop | Kabat | AbM | Chothia |
|---|---|---|---|
| L1 | L24-L34 | L24-L34 | L24-L34 |
| L2 | L50-L56 | L50-L56 | L50-L56 |
| L3 | L89-L97 | L89-L97 | L89-L97 |
| H1 | H31-H35B | H26-H35B | H26-H32 . . . 34 |
| | | (Kabat Numbering) | |
| H1 | H31-H35 | H26-H35 | H26-H32 |
| | | (Chothia Numbering) | |
| H2 | H50-H65 | H50-H58 | H52-H56 |
| H3 | H95-H102 | H95-H102 | H95-H102 |

As used herein, the term “constant region” and “constant domain” are interchangeable and have their common meanings in the art. The constant region is an antibody portion, e.g., a carboxyl terminal portion of a light and/or heavy chain which is not directly involved in binding of an antibody to antigen but which can exhibit various effector functions, such as interaction with the Fc receptor. The constant region of an immunoglobulin molecule generally has a more conserved amino acid sequence relative to an immunoglobulin variable domain.

As used herein, the term “heavy chain” when used in reference to an antibody can refer to any distinct type, e.g., alpha (α), delta (δ), epsilon (ε), gamma (γ), and mu (μ), based on the amino acid sequence of the constant domain, which give rise to IgA, IgD, IgE, IgG, and IgM classes of antibodies, respectively, including subclasses of IgG, e.g., IgG1, IgG2, IgG3, and IgG4. Heavy chain amino acid sequences are well known in the art. In some aspects of the present disclosure, the heavy chain is a human heavy chain.

As used herein, the term “light chain” when used in reference to an antibody can refer to any distinct type, e.g., kappa (κ) or lambda (λbased on the amino acid sequence of the constant domains. Light chain amino acid sequences are well known in the art. In some aspects of the present disclosure, the light chain is a human light chain.

As used herein, the terms “Programmed Death 1,” “Programmed Cell Death 1,” “Protein PD-1,” “PD-1,” “PD1,” “PDCD1,” “hPD-1” and “hPD-I” are used interchangeably. The complete PD-1 sequence can be found under NCBI Reference Sequence: N_012110.1. The amino acid sequence of the human PD-1 protein is:

(SEQ ID NO: 17)
MQIPQAPWPVVWAVLQLGWRPGWFLDSPDRPWNPPTFSPALLVVTEGDNA
TFTCSFSNTSESFVLNWYRMSPSNQTDKLAAFPEDRSQPGQDCRFRVTQL
PNGRDFHMSVVRARRNDSGTYLCGAISLAPKAQIKESLRAELRVTERRAE
VPTAHPSPSPRPAGQFQTLVVGVVGGLLGSLVLLVWVLAVICSRAARGTI
GARRTGQPLKEDPSAVPVFSVDYGELDFQWREKTPEPPVPCVPEQTEYAT
IVFPSGMGTSSPARRGSADGPRSAQPLRPEDGHCSWPL.

Programmed Death-1 (“PD-1”) is an approximately 31 kD type I membrane protein member of the extended CD28/CTLA-4 family of T cell regulators (see, Ishida, Y. et al. (1992) Induced Expression Of PD-1, A Novel Member Of The Immunoglobulin Gene Superfamily, Upon Programmed Cell Death,” EMBO J. 11:3887-3895.

PD-1 is expressed on activated T cells, B cells, and monocytes (Agata, Y. et al. (1996) “Expression of the PD-1 Antigen on the Surface of Stimulated Mouse T and B Lymphocytes,” Int. Immunol. 8(5):765-772; Martin-Orozco, N. et al. (2007) “Inhibitory Costimulation and Anti-Tumor Immunity,” Semin. Cancer Biol. 17(4):288-298). PD-1 is a receptor responsible for down-regulation of the immune system following activation by binding of PDL-1 or PDL-2 (Martin-Orozco, N. et al. (2007) “Inhibitory Costimulation and Anti-Tumor Immunity,” Semin. Cancer Biol. 17(4):288-298) and functions as a cell death inducer (Ishida, Y. et al. (1992) “Induced Expression of PD-1, A Novel Member of The Immunoglobulin Gene Superfamily, Upon Programmed Cell Death,” EMBO J. 11: 3887-3895; Subudhi, S. K. et al. (2005) “The Balance of Immune Responses: Costimulation Verse Coinhibition,” J. Molec. Med. 83: 193-202). This process is exploited in many tumors via the over-expression of PD-L1, leading to a suppressed immune response.

PD-1 is a well-validated target for immune mediated therapy in oncology, with positive results from clinical trials in the treatment of melanoma and non-small cell lung cancers (NSCLC), among others. Antagonistic inhibition of the PD-1/PD-L-1 interaction increases T-cell activation, enhancing recognition and elimination of tumour cells by the host immune system. The use of anti-PD-1 antibodies to treat infections and tumors and enhance an adaptive immune response has been proposed (see, U.S. Pat. Nos. 7,521,051; 7,563,869; 7,595,048).

Programmed Death Ligand 1 (PD-L1) is also part of a complex system of receptors and ligands that are involved in controlling T-cell activation. In normal tissue, PD-L1 is expressed on T cells, B cells, dendritic cells, macrophages, mesenchymal stem cells, bone marrow-derived mast cells, as well as various non-hematopoietic cells. Its normal function is to regulate the balance between T-cell activation and tolerance through interaction with its two receptors: programmed death 1 (also known as PD-1 or CD279) and CD80 (also known as B7-1 or B7.1). PD-L1 is also expressed by tumors and acts at multiple sites to help tumors evade detection and elimination by the host immune system. PD-L1 is expressed in a broad range of cancers with a high frequency. In some cancers, expression of PD-L1 has been associated with reduced survival and unfavorable prognosis. Antibodies that block the interaction between PD-L1 and its receptors are able to relieve PD-L1-dependent immunosuppressive effects and enhance the cytotoxic activity of antitumor T cells in vitro. Durvalumab is a human monoclonal antibody directed against human PD-L1 that is capable of blocking the binding of PD-L1 to both the PD-1 and CD80 receptors. The use of anti-PD-L1 antibodies to treat infections and tumors and enhance an adaptive immune response has been proposed (see, U.S. Pat. Nos. 8,779,108 and 9,493,565 incorporated herein by reference in their entirety).

As used herein, the terms “Cytotoxic T Lymphocyte associated Antigen-4,” “CTLA-4,” “CD152” and “hCTLA-4” are used interchangeably, and include variants, isoforms, species homologs of human CTLA-4. The complete CTLA-4 sequence can be found under NCBI Reference Sequence: NG_011502.1. The amino acid sequence of the human CTLA-4 protein is:

(SEQ ID NO: 18)
MACLGFQRHKAQLNLATRTWPCTLLFFLLFIPVFCKAMHVAQPAVVLASS
RGIASFVCEYASPGKATEVRVTVLRQADSQVTEVCAATYMMGNELTFLDD
SICTGTSSGNQVNLTIQGLRAMDTGLYICKVELMYPPPYYLGIGNGTQIY
VIDPEPCPDSDFLLWILAAVSSGLFFYSFLLTAVSLSKMLKKRSPLTTGV
YVKMPPTEPECEKQFQPYFIPIN.

Cytotoxic T-lymphocyte-associated protein 4 (CTLA-4) is expressed on activated T cells and serves as a co-inhibitor to keep T-cell responses in check following CD28-mediated T cell activation. CTLA-4 is believed to regulate the amplitude of the early activation of naive and memory T cells following TCR engagement and to be part of a central inhibitory pathway that affects both antitumor immunity and autoimmunity. CTLA-4 is expressed exclusively on T cells, and the expression of its ligands CD80 (B7.1) and CD86 (B7.2), is largely restricted to antigen-presenting cells, T cells, and other immune mediating cells. Antagonistic anti-CTLA-4 antibodies that block the CTLA-4 signaling pathway have been reported to enhance T-cell activation. One such antibody, ipilimumab, was approved by the FDA in 2011 for the treatment of metastatic melanoma. The use of anti-CTLA-4 antibodies to treat infections and tumors and up-modulate an adaptive immune response has been proposed (see, U.S. Pat. Nos. 6,682,736; 7,109,003; 7,132,281; 7,411,057; 7,824,679; 8,143,379 7,807,797; 8,491,895; 8,883,984; and US Publication No. 20150104409, incorporated herein by reference in their entireties).

The term “chimeric” antibodies or antigen-binding fragments thereof refers to antibodies or antigen-binding fragments thereof wherein the amino acid sequence is derived from two or more species. Typically, the variable region of both light and heavy chains corresponds to the variable region of antibodies or antigen-binding fragments thereof derived from one species of mammals (e.g. mouse, rat, rabbit, etc.) with the desired specificity, affinity, and capability while the constant regions are homologous to the sequences in antibodies or antigen-binding fragments thereof derived from another (usually human) to avoid eliciting an immune response in that species.

The term “humanized” antibody or antigen-binding fragment thereof refers to forms of non-human (e.g. murine) antibodies or antigen-binding fragments that are specific immunoglobulin chains, chimeric immunoglobulins, or fragments thereof that contain minimal non-human (e.g., murine) sequences. Typically, humanized antibodies or antigen-binding fragments thereof are human immunoglobulins in which residues from the complementary determining region (CDR) are replaced by residues from the CDR of a non-human species (e.g. mouse, rat, rabbit, hamster) that have the desired specificity, affinity, and capability (“CDR grafted”) (Jones et al., Nature 321:522-525 (1986); Riechmann et al., Nature 332:323-327 (1988); Verhoeyen et al., Science 239:1534-1536 (1988)). In some instances, certain Fv framework region (FR) residues of a human immunoglobulin are replaced with the corresponding residues in an antibody or fragment from a non-human species that has the desired specificity, affinity, and capability. The humanized antibody or antigen-binding fragment thereof can be further modified by the substitution of additional residues either in the Fv framework region and/or within the non-human CDR residues to refine and optimize antibody or antigen-binding fragment thereof specificity, affinity, and/or capability. In general, the humanized antibody or antigen-binding fragment thereof will comprise variable domains containing all or substantially all of the CDR regions that correspond to the non-human immunoglobulin whereas all or substantially all of the FR regions are those of a human immunoglobulin consensus sequence. The humanized antibody or antigen-binding fragment thereof can also comprise at least a portion of an immunoglobulin constant region or domain (Fc), typically that of a human immunoglobulin. Examples of methods used to generate humanized antibodies are described in U.S. Pat. No. 5,225,539; Roguska et al., Proc. Natl. Acad. Sci., USA, 91(3):969-973 (1994), and Roguska et al., Protein Eng. 9(10):895-904 (1996). In some aspects of the present disclosure, a “humanized antibody” is a resurfaced antibody.

The term “human” antibody or antigen-binding fragment thereof means an antibody or antigen-binding fragment thereof having an amino acid sequence derived from a human immunoglobulin gene locus, where such antibody or antigen-binding fragment is made using any technique known in the art. This definition of a human antibody or antigen-binding fragment thereof includes intact or full-length antibodies and fragments thereof.

“Binding affinity” generally refers to the strength of the sum total of non-covalent interactions between a single binding site of a molecule (e.g., an antibody or antigen-binding fragment thereof) and its binding partner (e.g., an antigen). Unless indicated otherwise, as used herein, “binding affinity” refers to intrinsic binding affinity which reflects a 1:1 interaction between members of a binding pair (e.g., antibody or antigen-binding fragment thereof and antigen). The affinity of a molecule X for its partner Y can generally be represented by the dissociation constant (KD). Affinity can be measured and/or expressed in a number of ways known in the art, including, but not limited to, equilibrium dissociation constant (KD), and equilibrium association constant (KA). The KD is calculated from the quotient of k_{off}/kₒₙ, whereas KA is calculated from the quotient of k_{off}/kₒₙ. Kₒₙ refers to the association rate constant of, e.g., an antibody or antigen-binding fragment thereof to an antigen, and k_{off} refers to the dissociation of, e.g., an antibody or antigen-binding fragment thereof from an antigen. The kₒₙ and k_{off} can be determined by techniques known to one of ordinary skill in the art, such as BIAcore® or KinExA.

As used herein, an “epitope” is a term in the art and refers to a localized region of an antigen to which an antibody or antigen-binding fragment thereof can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In some aspects of the present disclosure, the epitope to which an antibody or antigen-binding fragment thereof specifically binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization can be accomplished using any of the known methods in the art (e.g., Giegé R et al., (1994) Acta Crystallogr D Biol Crystallogr 50(Pt 4): 339-350; McPherson A (1990) Eur J Biochem 189: 1-23; Chayen N E (1997) Structure 5: 1269-1274; McPherson A (1976) J Biol Chem 251: 6300-6303). Antibody/antigen-binding fragment thereof: antigen crystals can be studied using well known X-ray diffraction techniques and can be refined using computer software such as X-PLOR (Yale University, 1992, distributed by Molecular Simulations, Inc.; see, e.g., Meth Enzymol (1985) volumes 114 & 115, eds Wyckoff H W et al.,; U.S. 2004/0014194), and BUSTER (Bricogne G (1993) Acta Crystallogr D Biol Crystallogr 49(Pt 1): 37-60; Bricogne G (1997) Meth Enzymol 276A: 361-423, ed Carter C W; Roversi P et al., (2000) Acta Crystallogr D Biol Crystallogr 56(Pt 10): 1316-1323). Mutagenesis mapping studies can be accomplished using any method known to one of skill in the art. See, e.g., Champe M et al., (1995) J Biol Chem 270: 1388-1394 and Cunningham B C & Wells J A (1989) Science 244: 1081-1085 for a description of mutagenesis techniques, including alanine scanning mutagenesis techniques.

An antibody that “binds to the same epitope” as a reference antibody refers to an antibody that binds to the same amino acid residues as the reference antibody. The ability of an antibody to bind to the same epitope as a reference antibody can determined by a hydrogen/deuterium exchange assay (see Coales et al. Rapid Commun. Mass Spectrom. 2009; 23: 639-647) or x-ray crystallography.

An antibody is said to “competitively inhibit” or “cross compete” with binding of a reference antibody to a given epitope if it preferentially binds to that epitope or an overlapping epitope to the extent that it blocks, to some degree, binding of the reference antibody to the epitope. Competitive inhibition can be determined by any method known in the art, for example, competition ELISA assays. An antibody can be said to competitively inhibit binding of the reference antibody to a given epitope by at least 90%, at least 80%, at least 70%, at least 60%, or at least 50%.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cell or compositions include those which have been purified to a degree that they are no longer in a form in which they are found in nature. In some aspects of the present disclosure, an antibody, polynucleotide, vector, cell, or composition which is isolated is substantially pure. As used herein, “substantially pure” refers to material which is at least 50% pure (i.e., free from contaminants), at least 90% pure, at least 95% pure, at least 98% pure, or at least 99% pure.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this disclosure are based upon antibodies, in some aspects of the present disclosure, the polypeptides can occur as single chains or associated chains.

As used herein, the term “MEDI5752” refers to an anti-PD-1/CTLA-4 bispecific antibody that comprises the light chain of SEQ ID NO:1 and the heavy chain of SEQ ID NO:2 (PD-1) and the light chain of SEQ ID NO:3 and the heavy chain of SEQ ID NO:4 (CTLA-4). MEDI5752 is disclosed in U.S. Pat. No. 10,457,732, which is herein incorporated by reference in its entirety.

As used herein, the term “pharmaceutical formulation” refers to a preparation which is in such form as to permit the biological activity of the active ingredient to be effective, and which contains no additional components which are unacceptably toxic to a subject to which the formulation would be administered. The formulation can be sterile.

The terms “administer,” “administering,” “administration,” and the like, as used herein, refer to methods that can be used to enable delivery of a drug, e.g., an anti-PD1/CTLA-4 antibody or antigen-binding fragment thereof to the desired site of biological action (e.g., intravenous administration). Administration techniques that can be employed with the agents and methods described herein are found in e.g., Goodman and Gilman, The Pharmacological Basis of Therapeutics, current edition, Pergamon; and Remington's, Pharmaceutical Sciences, current edition, Mack Publishing Co., Easton, Pa.

As used herein, the terms “combination” or “administered in combination” means that an antibody or antigen binding fragment thereof described herein can be administered with one or more additional therapeutic agents. In some aspects, an antibody or antigen binding fragment thereof can be administered with one or more additional therapeutic agents either simultaneously or sequentially. In some aspects, an antibody or antigen binding fragment thereof described herein can be administered with one or more additional therapeutic agent in the same or in different compositions.

As used herein, the terms “subject” and “patient” are used interchangeably. The subject can be an animal. In some aspects of the present disclosure, the subject is a mammal such as a non-human animal (e.g., cow, pig, horse, cat, dog, rat, mouse, monkey or other primate, etc.). In some aspects of the present disclosure, the subject is a cynomolgus monkey. In some aspects of the present disclosure, the subject is a human.

The term “therapeutically effective amount” refers to an amount of a drug, e.g., an anti-PD1/CTLA-4 antibody or antigen-binding fragment thereof, effective to treat a disease or disorder in a subject. Terms such as “treating,” “treatment,” “to treat,” “alleviating,” and “to alleviate” refer to therapeutic measures that cure, slow down, lessen symptoms of, and/or halt progression of a pathologic condition or disorder. Thus, those in need of treatment include those already diagnosed with or suspected of having the disorder.

As used in the present disclosure and claims, the singular forms “a,” “an,” and “the” include plural forms unless the context clearly dictates otherwise.

It is understood that wherever aspects of the present disclosure are described herein with the language “comprising,” otherwise analogous aspects described in terms of “consisting of” and/or “consisting essentially of” are also provided.

Unless specifically stated or obvious from context, as used herein, the term “or” is understood to be inclusive. The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, the terms “about” and “approximately,” when used to modify a numeric value or numeric range, indicate that deviations of 5% to 10% above and 5% to 10% below the value or range remain within the intended meaning of the recited value or range.

Any compositions or methods provided herein can be combined with one or more of any of the other compositions and methods provided herein.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

### 5.2 Methods of the Invention

In one aspect, the present invention is directed to a method for treating renal cell carcinoma or non-small cell lung cancer in a subject in need thereof. A therapy comprising an anti-PD-1/CTLA-4 bispecific antibody or antigen-binding fragment thereof results in better therapeutic outcomes (e.g., objective response rate and disease control rate) for afflicted subjects.

In one aspect, the invention includes a method of selecting a renal cell carcinoma or non-small cell lung cancer in a human patient for immunotherapy, comprising determining the level of PD-L1 expression in a tumor sample. In some aspects, the tumor sample is PD-L1 positive. In some aspects, the tumor sample is PD-L1 negative.

In some aspects, the invention includes a method of inhibiting growth of a renal cell carcinoma in a human patient, comprising administering to the patient an anti-PD-1/CTLA-4 bispecific antibody. In one aspect, the invention includes a method of treating a renal cell carcinoma in a human patient, comprising administering to the patient an anti-PD-1/CTLA-4 bispecific antibody. In some aspects, the bispecific antibody is MEDI5752.

In some aspects, the invention includes a method of inhibiting growth of a non-small cell lung cancer in a human patient, comprising administering to the patient an anti-PD-1/CTLA-4 bispecific antibody. In one aspect, the invention includes a method of treating a non-small cell lung cancer in a human patient, comprising administering to the patient an anti-PD-1/CTLA-4 bispecific antibody. In some aspects, the bispecific antibody is MEDI5752.

In some aspects, the method of treating renal cell carcinoma or non-small cell lung cancer comprises administering to the subject about 100 mg to about 1500 mg of a bispecific antibody (for example, MEDI5752) or antigen-binding fragment thereof. In some aspects, the method comprises administering about 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, about 1000 mg, about 1010 mg, about 1020 mg, about 1030 mg, about 1040 mg, about 1050 mg, about 1060 mg, about 1070 mg, about 1080 mg, about 1090 mg, about 1100 mg, about 1120 mg, about 1130 mg, about 1140 mg, about 1150 mg, about 1160 mg, about 1170 mg, about 1180 mg, about 1190 mg, about 1200 mg, about 1250 mg, about 1300 mg, about 1350 mg, about 1400 mg, about 1450 mg, or about 1500 mg. In some aspects, the method comprises administering a priming dose at about 750 mg, about 1000 mg, about 1250 mg, or about 1500 mg followed by a maintenance dose of about 225 mg, about 500 mg, about 750 mg, 1000 mg, or about 1250 mg. In some aspects, the method comprises administering a priming dose of about 750 mg and a maintentance dose of about 225 mg. In some aspects, the method comprises administering a priming dose of about 750 mg and a maintentance dose of about 500 mg. In some aspects, the method comprises administering a priming dose of about 750 mg and a maintentance dose of about 750 mg. In some aspects, the method comprises administering a priming dose of about 750 mg and a maintentance dose of about 1000 mg. In some aspects, the method comprises administering a priming dose of about 750 mg and a maintentance dose of about 1250 mg. In some aspects, the method comprises administering a priming dose of about 750 mg and a maintentance dose of about 225 mg. In some aspects, the method comprises administering a priming dose of about 750 mg and a maintentance dose of about 500 mg. In some aspects, the method comprises administering a priming dose of about 750 mg and a maintentance dose of about 750 mg. In some aspects, the method comprises administering a priming dose of about 750 mg and a maintentance dose of about 1000 mg. In some aspects, the method comprises administering a priming dose of about 750 mg and a maintentance dose of about 1250 mg. In some aspects, the method comprises administering a priming dose of about 1000 mg and a maintentance dose of about 225 mg. In some aspects, the method comprises administering a priming dose of about 1000 mg and a maintentance dose of about 500 mg. In some aspects, the method comprises administering a priming dose of about 1000 mg and a maintentance dose of about 750 mg. In some aspects, the method comprises administering a priming dose of about 1000 mg and a maintentance dose of about 1000 mg. In some aspects, the method comprises administering a priming dose of about 1000 mg and a maintentance dose of about 1250 mg. In some aspects, the method comprises administering a priming dose of about 1250 mg and a maintentance dose of about 225 mg. In some aspects, the method comprises administering a priming dose of about 1250 mg and a maintentance dose of about 500 mg. In some aspects, the method comprises administering a priming dose of about 1250 mg and a maintentance dose of about 750 mg. In some aspects, the method comprises administering a priming dose of about 1250 mg and a maintentance dose of about 1250 mg. In some aspects, the method comprises administering a priming dose of about 1250 mg and a maintentance dose of about 1250 mg. In some aspects, the method comprises administering a priming dose of about 1500 mg and a maintentance dose of about 225 mg. In some aspects, the method comprises administering a priming dose of about 1500 mg and a maintentance dose of about 500 mg. In some aspects, the method comprises administering a priming dose of about 1500 mg and a maintentance dose of about 750 mg. In some aspects, the method comprises administering a priming dose of about 1500 mg and a maintentance dose of about 1500 mg. In some aspects, the method comprises administering a priming dose of about 1500 mg and a maintentance dose of about 1250 mg. In some aspects, the dose of MEDI5752 administered can be reduced with continuing administration.

In some aspects, the method of treating renal cell carcinoma or non-small cell lung cancer comprises administering to the subject about 500 mg or 750 mg of a bispecific antibody (for example, MEDI5752) or antigen-binding fragment thereof. In some aspects, the method of treating renal cell carcinoma or non-small cell lung cancer comprises administering about 1000 mg of the bispecific antibody or antigen-binding fragment thereof. In some aspects, the method of treating renal cell carcinoma or non-small cell lung cancer comprises administering about 1125 mg of the bispecific antibody or antigen-binding fragment thereof.

In some aspects, a dose of the bispecific antibody or antigen-binding fragment thereof is administered to the subject once per treatment cycle. In some aspects, a treatment cycle is three weeks. In some aspects, a dose of the bispecific antibody or antigen-binding fragment thereof is administered every three weeks for about 12 months, about 24 months, about 36 months, or about 48 months.

In some aspects, the bispecific antibody or antigen-binding fragment thereof is administered in combination with one or more chemotherapeutic agents. In some aspects, the chemotherapeutic agent is carboplatin. In some aspects, the chemotherapeutic agent is pemetrexed. In some aspects, the chemotherapeutic agents is axitinib.

In some aspects, the bispecific antibody or antigen-binding fragment thereof is administered in combination with more than one chemotherapeutic agent. In some aspects, the method of treating non-small cell lung cancer further comprises administration of the chemotherapeutic agents carboplatin and pemetrexed. In some aspects, the method of treating non-small cell lung cancer further comprises administration of the chemotherapeutic agents carboplatin and paclitaxel. one or more chemotherapeutic agents are carboplatin and nab-paclitaxel.

In some aspects, carboplatin is administered at a dose between about AUC 4 mg/mL·min and AUC 6 mg/mL·min. In some aspects, pemetrexed is administered at a dose between about 400 mg/m² and 600 mg/m². In some aspects, nab-paclitaxel is administered at a dose between about 50 mg/m² and 150 mg/m². In some aspects, paclitaxel is administered at a dose between about 150 mg/m² and 250 mg/m². In some aspects, axitinib is administered at a dose between about 4 mg and 6 mg. In some aspects, lenvatinib is administered at a dose between about 8 mg and 20 mg. In some aspects, lenvatinib is administered at a dose of about 8 mg, 10 mg, 12 mg, 14 mg, 16 mg, 18 mg, or 20 mg. In some aspects, lenvatinib is administered once daily.

In some aspects, the method of treating non-small cell lung cancer comprises administration of a bispecific antibody (for example, MEDI5752) or antigen-binding fragment thereof at a dose of about 500 mg or 750 mg, carboplatin is administered at a dose of AUC 5 mg/mL·min and pemetrexed is administered at a dose of 500 mg/m². In some aspects, the carboplatin and pemetrexed are administered every three weeks for four cycles (i.e., twelve weeks). In some aspects, the administration is followed by maintenance dosing. Maintenance dosing involves administering the bispecific antibody or antigen-binding fragment thereof in combination with pemetrexed once every three weeks. In some aspects, the maintenance dosing can be indefinite.

In some aspects, the method of treating renal cell carcinoma further comprises administration of the chemotherapeutic agent axitinib. In some aspects, the chemotherapeutic agent, (e.g., axitinib) is orally administered at a dose of 5 mg twice daily, from day −7 to day −1, prior to administration of the bispecific antibody (for example, MEDI5752) or antigen-binding fragment thereof. In such aspects, the bispecific antibody or antigen-binding fragment thereof is administered on day 1. In some aspects, the axitinib is administered at a dose of 5 mg twice daily and the bispecific antibody or antigen-binding fragment thereof is administered at a dose of 1500 mg once every three weeks. In such aspects, the axitinib and the bispecific antibody or antigen-binding fragment thereof are administered for about 12 months, about 24 months, or about 36 months. In some aspects, the axitinib and the bispecific antibody or antigen-binding fragment thereof are administered for about 12 months. In some aspects, the axitinib and the bispecific antibody or antigen-binding fragment thereof are administered for about 24 months. In some aspects, the axitinib and the bispecific antibody or antigen-binding fragment thereof are administered for about 36 months. In some aspects, the axitinib and the bispecific antibody or antigen-binding fragment thereof are administered for about 48 months.

In some aspects, the method of treating renal cell carcinoma further comprises administration of the chemotherapeutic agent lenvatinib. In some aspects, the chemotherapeutic agent, (e.g., lenvatinib) is orally administered at a dose of 14 mg or 18 mg once daily, from day −7 to day −1, prior to administration of the bispecific antibody (for example, MEDI5752) or antigen-binding fragment thereof. In such aspects, the bispecific antibody or antigen-binding fragment thereof is administered on day 1. In some aspects, the lenvatinib is administered at a dose of 14 mg or 18 mg once daily and the bispecific antibody or antigen-binding fragment thereof is administered at a dose of 500 mg or 750 mg once every three or four weeks. In such aspects, the lenvatinib and the bispecific antibody or antigen-binding fragment thereof are administered for about 12 months, about 24 months, or about 36 months. In some aspects, the lenvatinib and the bispecific antibody or antigen-binding fragment thereof are administered for about 12 months. In some aspects, the lenvatinib and the bispecific antibody or antigen-binding fragment thereof are administered for about 24 months. In some aspects, the lenvatinib and the bispecific antibody or antigen-binding fragment thereof are administered for about 36 months. In some aspects, the lenvatinib and the bispecific antibody or antigen-binding fragment thereof are administered for about 48 months.

In some aspects, the method of treating non-small cell lung cancer comprises administration of the bispecific antibody (for example, MEDI5752) or antigen-binding fragment thereof at a dose of 500 or 750 mg every three weeks, carboplatin is administered at a dose of AUC 5 mg/mL·min every three weeks for 4 cycles (i.e., twelve weeks), and pemetrexed is administered at a dose of 500 mg/m² every three weeks. In such aspects, the bispecific antibody or antigen-binding fragment thereof, the carboplatin, and the pemetrexed are administered for about 12 months, about 24 months, about 36 months, or about 48 months. In some aspects, the bispecific antibody or antigen-binding fragment thereof, the carboplatin, and the pemetrexed are administered for about 12 months. In some aspects, the bispecific antibody or antigen-binding fragment thereof, the carboplatin, and the pemetrexed are administered for about 24 months. In some aspects, the bispecific antibody or antigen-binding fragment thereof, the carboplatin, and the pemetrexed are administered for about 36 months or longer.

In some aspects, the method of treating non-small cell lung cancer comprises administration the bispecific antibody (for example, MEDI5752) or antigen-binding fragment thereof in combination with carboplatin and paclitaxel. In some aspects, the bispecific antibody or antigen-binding fragment thereof is administered at a dose of 500 or 750 mg every three weeks, the carboplatin is administered at a dose of AUC 5 mg/mL·min every three weeks for 4 cycles and the paclitaxel is administered at a dose of 200 mg/m² body surface area (BSA) on days 1, 8, and 15 of every three week cycle for 4 cycles. In such aspects, the carboplatin is administered immediately after paclitaxel is administered. In some aspects, the bispecific antibody or antigen-binding fragment thereof, the carboplatin, and the paclitaxel are administered for about 12 months, about 24 months, or about 36 months. In some aspects, the bispecific antibody or antigen-binding fragment thereof, the carboplatin, and the paclitaxel are administered for about 12 months. In some aspects, the bispecific antibody or antigen-binding fragment thereof, the carboplatin, and the paclitaxel are administered for about 24 months. In some aspects, the bispecific antibody or antigen-binding fragment thereof, the carboplatin, and the nab-paclitaxel are administered for about 36 months or longer.

In some aspects, the method of treating non-small cell lung cancer comprises administration the bispecific antibody (for example, MEDI5752) or antigen-binding fragment thereof in combination with carboplatin and Nab-paclitaxel. In some aspects, the bispecific antibody or antigen-binding fragment thereof is administered at a dose of 500 or 750 mg every three weeks, the carboplatin is administered at a dose of AUC 5 mg/mL·min every three weeks for 4 cycles and the Nab-paclitaxel is administered at a dose of 100 mg/m² body surface area (BSA) on days 1, 8, and 15 of every three week cycle for 4 cycles. In such aspects, the carboplatin is administered immediately after nab-paclitaxel is administered. In some aspects, the bispecific antibody or antigen-binding fragment thereof, the carboplatin, and the nab-paclitaxel are administered for about 12 months, about 24 months, or about 36 months. In some aspects, the bispecific antibody or antigen-binding fragment thereof, the carboplatin, and the nab-paclitaxel are administered for about 12 months. In some aspects, the bispecific antibody or antigen-binding fragment thereof, the carboplatin, and the nab-paclitaxel are administered for about 24 months. In some aspects, the bispecific antibody or antigen-binding fragment thereof, the carboplatin, and the nab-paclitaxel are administered for about 36 months or longer.

In some aspects, the invention includes a method for extending a progression-free survival period for over 12 months in a human patient afflicted with renal cell carcinoma or non-small cell lung cancer comprising administering to the patient an immunotherapy disclosed herein, wherein the patient demonstrates progression-free survival for over 12 months. In some aspects, the progression-free survival of the patient can be extended, after the administration, for over about 13 months, about 14 months, about 15 months, about 16 months, about 17 months, about 18 months, about 2 years, about 3 years, about 4 years, about 5 years, about 6 years, about 7 years, about 8 years, about 9 years, or about 10 years as compared to standard of care therapy.

In some aspects, the invention includes a method for extending the overall response rate (ORR) that is at least about 10%, 15%, 20%, 30%, 40%, 50%, 60%, 70%, 75% longer or higher as compared to standard of care therapy.

In some aspects, the invention includes a method for extending the overall survival that is at least about 10%, 15%, 20%, 30%, 40%, 50%, 60%, 70%, 75% longer or higher as compared to standard of care therapy. In some aspects, the overall survival for a patient treated with a method of the invention is at least about 12, 13, 14, 15, 16, 17, 18, 19, 20, 21, 22, 23, 24 or more months.

In still other aspects, the invention is includes a method for reducing a tumor size at least by 10% in a human patient afflicted with renal cell carcinoma or non-small cell lung cancer comprising administering an immunotherapy disclosed herein, wherein the administration reduces the tumor size at least about 10%, about 20%, about 30%, about 40%, about 50%, about 60%, about 70%, about 80%, about 90%, or 100% compared to the tumor size prior to the administration. In some aspects, the method comprises identifying the patient as having a PD-L1 positive tumor prior to the administration.

In some aspects, the invention includes a method for increasing an objective response rate to be higher than 15% in a patient population. In some aspects, objective response rate is higher than 10%, 15%, 20%, 25%, 30%, 35%, 40%, 45%, 50%, 55%, 60%, 65%, 70%, 75% or higher. In some aspects, the method comprises identifying the patient as having a PD-L1 positive tumor prior to the administration.

In some aspects, each patient in the methods experiences (i) extended progression-free survival for over 12 months, (ii) tumor size reduction at least about 10%, about 20%, about 30%, about 40%, or about 50% compared to the tumor size prior to the administration, or (iii) both.

The methods of the invention, as a result of the administration of an immunotherapy disclosed herein, can treat the renal cell carcinoma or non-small cell lung cancer, reduce the tumor size, inhibit growth of the tumor, eliminate the tumor from the patient, prevent a relapse of a tumor, induce a remission in a patient, or any combination thereof. In certain aspects, the administration of an immunotherapy disclosed herein induces a complete response. In other aspects, the administration of the immunotherapy disclosed herein induces a partial response. In some aspects, the responses are evaluated according to RECIST. In some aspects, the responses are evaluated according to iRECIST. In some aspects, the responses are evaluated according to pathological responses.

In some aspects, the PD-L1 positive tumor comprises at least about 1%, at least about 2%, at least about 3%, at least about 4%, at least about 5%, at least about 7%, at least about 10%, at least about 15%, at least about 20%, at least about 25%, at least about 30%, at least about 40%, at least about 50%, at least about 60%, at least about 70%, at least about 80%, at least about 90%, or 100% cells expressing PD-L1. In some aspects, the PD-L1 positive tumor comprises about 1% to about 49% cells expressing PD-L1. In some aspects, the PD-L1 positive tumor comprises about ≥50% cells expressing PD-L1.

In some aspects, the PD-L1 positive tumor comprises less than about 1% cells expressing PD-L1. In some aspects, the tumor comprises 0% cells expressing PD-L1. In some aspects, PD-L1 positive tumor percentages can be determined by assays known to one skilled in the art. In some aspects, the Ventana PD-L1 (SP263) can be used.

In some aspects, PD-L1 expression is determined by receiving the results of an assay capable of determining PD-L1 expression.

In some aspects, a method of the invention alters the frequency of activated/proliferating and effector T cells. In some aspects, the T cells are measured by flow cytometry-based assays or immunohistochemistry.

In some aspects, a method of the invention alters protein or gene expression of biomarkers such as but not limited to PD-1, PD-L1, CTLA-4, CD8, and IFN-γ.

In order to assess gene or protein expression (for example, PD-L1), in one aspects, a test tissue sample is obtained from the patient who is in need of the therapy. In some aspects, a test tissue sample includes, but is not limited to, any clinically relevant tissue sample, such as a tumor biopsy, a core biopsy tissue sample, a fine needle aspirate, or a sample of bodily fluid, such as blood, plasma, serum, lymph, ascites fluid, cystic fluid, or urine. In some aspects, the test tissue sample is from a primary tumor. In some aspects, the test tissue sample is from a metastasis. In some aspects, test tissue samples are taken from a subject at multiple time points, for example, before treatment, during treatment, and/or after treatment. In some aspects, test tissue samples are taken from different locations in the subject, for example, a sample from a primary tumor and a sample from a metastasis in a distant location.

In some aspects, the test tissue sample is a paraffin-embedded fixed tissue sample. In some aspects, the test tissue sample is a formalin-fixed paraffin embedded (FFPE) tissue sample. In some aspects, the test tissue sample is a fresh tissue (e.g., tumor) sample. In some aspects, the test tissue sample is a frozen tissue sample. In some aspects, the test tissue sample is a fresh frozen (FF) tissue (e.g., tumor) sample. In some aspects, the test tissue sample is a cell isolated from a fluid. In some aspects, the test tissue sample comprises circulating tumor cells (CTCs). In some aspects, the test tissue sample comprises tumor-infiltrating lymphocytes (TILs). In some aspects, the test tissue sample comprises tumor cells and tumor-infiltrating lymphocytes (TILs). In some aspects, the test tissue sample comprises circulating lymphocytes. In some aspects, the test tissue sample is an archival tissue sample. In some aspects, the test tissue sample is an archival tissue sample with known diagnosis, treatment, and/or outcome history. In some aspects, the sample is a block of tissue. In some aspects, the test tissue sample is dispersed cells. In some aspects, the sample size is from about 1 cell to about 1×10⁶ cells or more. In some aspects, the sample size is about 1 cell to about 1×10⁵ cells. In some aspects, the sample size is about 1 cell to about 10,000 cells. In some aspects, the sample size is about 1 cell to about 1,000 cells. In some aspects, the sample size is about 1 cells to about 100 cells. In some aspects, the sample size is about 1 cell to about 10 cells. In some aspects, the sample size is a single cell.

In another aspect, the assessment of expression can be achieved without obtaining a test tissue sample. In some aspects, selecting a suitable patient includes (i) optionally providing a test tissue sample obtained from a patient with cancer of the tissue, the test tissue sample comprising tumor cells and/or tumor-infiltrating inflammatory cells; and (ii) assessing the proportion of cells in the test tissue sample that express the gene/protein of interest, based on an assessment that the proportion of cells in the test tissue sample is higher than a predetermined threshold level.

In certain aspects of any of the present methods, the proportion of cells that express PD-L1, is assessed by performing an assay to detect the presence of PD-L1 RNA. In further aspects, the presence of PD-L1 RNA is detected by RT-PCR, in situ hybridization or RNase protection. In some aspects, the presence of PD-L1 RNA is detected by an RT-PCR based assay. In some aspects, scoring the RT-PCR based assay comprises assessing the level of PD-L1 RNA expression in the test tissue sample relative to a predetermined level.

In other aspects, the proportion of cells that express a gene/protein of interest (for example PD-L1) is assessed by performing an assay to detect the presence of PD-L1 polypeptide. In further aspects, the presence of the polypeptide is detected by IHC, enzyme-linked immunosorbent assay (ELISA), in vivo imaging, or flow cytometry. In some aspects, protein expression is assayed by IHC.

### 5.3 Patient Populations

Provided herein are clinical methods for treating cancers (e.g., advanced renal cell carcinomas or non-small cell lung cancers) in human patients using any method disclosed herein, for example, a bispecific antibody (for example, MEDI5752) or antigen-binding fragment thereof, administered as a single agent or optionally in combination with one or more chemotherapeutic agents. In some aspects, the patient has an advanced solid tumor. In some aspects, the patient has not received prior immunotherapy exposure and has tumors that are either refractory to standard therapy or for which there is no standard therapy. In some aspects, the patient is immunotherapy naïve with advanced or metastatic solid tumors. In some aspects, the patient has advanced clear-cell renal cell carcinoma. In some aspects, the patient has first line Stage IIIB or IV, nonsquamous non-small cell lung cancer. In some aspects, the patient is eligible to receive platinum-based doublet chemotherapy.

In some aspects, the patient has squamous non-small cell lung cancer. In some aspects, the patient has nonsquamous non-small cell lung cancer. In some aspects, the patient has an advancing solid tumor.

### 5.4 Outcomes

A patient treated according to the methods disclosed herein preferably experience improvement in at least one sign of cancer. In one aspect, improvement is measured by a reduction in the quantity and/or size of measurable tumor lesions. In another aspect, lesions can be measured on chest x-rays or CT or MRI films. In another aspect, cytology or histology can be used to evaluate responsiveness to a therapy. In some aspects, tumor response to the administration of the bispecific antibody or antigen-binding fragment thereof can be determined by Investigator review of tumor assessments and defined by the RECIST v1.1 guidelines. Additional tumor measurements can be performed at the discretion of the Investigator or according to institutional practice.

In some aspects, the patient treated exhibits a complete response (CR), i.e., the disappearance of all target lesions. In some aspects, the patient treated exhibits a partial response (PR), i.e., at least a 30% decrease in the sum of the diameters of target lesions, taking as reference the baseline sum diameters. In some aspects, the patient treated exhibits progressive disease (PD), i.e., at least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. (Note: The appearance of one or more new lesions may be considered progression). In some aspects, the patient treated exhibits stable disease (SD), i.e., neither sufficient shrinkage to qualify for PR nor sufficient increase to qualify for PD, taking as reference the smallest sum of diameters while on study.

In another aspect, the patient treated experiences tumor shrinkage and/or decrease in growth rate, i.e., suppression of tumor growth. In some aspects, unwanted cell proliferation is reduced or inhibited. In some aspects, one or more of the following can occur: the number of cancer cells can be reduced; tumor size can be reduced; cancer cell infiltration into peripheral organs can be inhibited, retarded, slowed, or stopped; tumor metastasis can be slowed or inhibited; tumor growth can be inhibited; recurrence of tumor can be prevented or delayed; one or more of the symptoms associated with cancer can be relieved to some extent.

In other aspects, administration of a bispecific antibody or antigen-binding fragment thereof according to any of the methods provided herein produces at least one therapeutic effect selected from the group consisting of reduction in size of a tumor, reduction in number of metastatic lesions appearing over time, complete remission, partial remission, or stable disease.

In some aspects, one or more tumor biopsies can be used to determine tumor response to administration of a bispecific antibody or antigen-binding fragment thereof according to any of the methods provided herein. In some aspects, the sample is a formalin-fixed paraffin embedded (FFPE) sample. In some aspects, the sample is a fresh sample. Tumor samples (e.g., biopsies) can be used to identify predictive and/or pharmacodynamic biomarkers associated with immune and tumor microenvironment. Such biomarkers can be determined from assays including IHC, tumor mutation analysis, RNA analysis, and proteomic analyses. In certain aspects, expression of tumor biomarkers are detected by RT-PCR, in situ hybridization, RNase protection, RT-PCR-based assay, immunohistochemistry, enzyme linked immuosorbent assay, in vivo imaging, or flow cytometry.

### 5.5 Bispecific Antibodies and Antigen-Binding Fragments Thereof

Provided herein are methods of treating cancers in a subject (e.g., a human subject) comprising administering to the subject antibodies (e.g., bispecific, monoclonal antibodies, such as chimeric, humanized, or human antibodies) and antigen-binding fragments thereof which specifically bind to PD-1 and CTLA-4, (e.g., human PD-1 and CTLA-4). In some aspects, PD-1 and CTLA-4, (e.g., human PD-1 and CTLA-4) antibodies and antigen-binding fragments thereof that can be used in the methods provided herein include MEDI5752, a monovalent bispecific humanized immunoglobulin G1 (IgG1) monoclonal antibody (mAb) with an engineered fragment crystallizable (Fc) domain to reduce Fc effector function, that specifically binds PD-1 and CTLA-4.

MEDI5752 was constructed on the backbone of the DuetMab molecule. The DuetMab design is described in Mazor et al., MAbs. 7(2): 377-389, (2015 March-April 2015), which is hereby incorporated by reference in its entirety. The “DuetMab,” design includes knobs-into-holes (KIH) technology for heterodimerization of 2 distinct heavy chains and increases the efficiency of cognate heavy and light chain pairing by replacing the native disulfide bond in one of the CH1-CL interfaces with an engineered disulfide bond.

The Fc domain of MEDI5752 carries the triple mutations (TM) (L234F, L235E and P331S) designed to reduce Fc-mediated immune effector functions (Oganesyan et al, Acta Crystallogr D Biol Crystallogr, 2008, 64(Pt 6): 700-704). MEDI5752 includes anti-PD-1 and anti-CTLA-4 Fabs, engineered interchain disulfide in the anti-CTLA-4 CH1-CL interface and knob-into-hole IgG1-TM Fc. MEDI5752 includes a knob mutation in the heavy chain comprising a variable region that binds to CTLA-4 and the hole mutation in the heavy chain comprising a variable region that binds to PD-1.

MEDI5752 is described in U.S. Pat. No. 10,457,732, which is incorporated by reference herein in its entirety.

In some aspects of the present disclosure, a bispecific antibody or antigen-binding fragment thereof for use in the methods described herein specifically binds to human PD-1 and human CTLA-4 and comprises the six CDRs of the MEDI5752 antibody listed as provided in Table 1.

TABLE 1
VH CDR Amino Acid Sequences¹

| | VH CDR1 | | |
|---|---|---|---|
| Anti- | (SEQ ID | VH CDR2 | VH CDR3 |
| body | NO:) | (SEQ ID NO:) | (SEQ ID NO:) |
| MEDI | GFTFSDYGMH | YISSGSYTIYSAD | RAPNSFYEYYFDY |
| 5752 | (SEQ ID | SVKG | (SEQ ID |
| PD-1 | NO: 8) | (SEQ ID NO: 9) | NO: 10) |
| MEDI | GFTFSSYGMH | VIWYDGSNKYYAD | DPRGATLYYYYYG |
| 5752 | (SEQ ID | SVKG | MDV |
| CTLA-4 | NO: 14) | (SEQ ID NO: 15) | (SEQ ID |
| | | | NO: 16) |

¹The VH CDRs in Table 1 are determined according to Kabat.

TABLE 2
VL CDR Amino Acid Sequences²

| | | VL CDR2 | VL CDR3 |
|---|---|---|---|
| Anti- | VL CDR1 | (SEQ ID | (SEQ ID |
| body | (SEQ ID NO:) | NO:) | NO:) |
| MEDI | SASSKHTNLYWSRHM | TSNRAT | QQWSSNP |
| 5752 | YWY | (SEQ ID | (SEQ ID |
| PD-1 | (SEQ ID NO: 5) | NO: 6) | NO: 7) |
| MEDI | RASQSINSYLD | AASSLQS | QQYYSTP |
| 5752 | (SEQ ID NO: 11) | (SEQ ID | (SEQ ID |
| CTLA- | | NO: 12) | NO: 13) |
| 4 | | | |

²The VL CDRs in Table 2 are determined according to Kabat.

In some aspects of the present disclosure, a bispecific antibody or antigen-binding fragment thereof for use in the methods described herein specifically binds to human PD-1 and CTLA-4 and comprises the variable heavy chain (VH) and variable light chain (VL) of the MEDI5752 antibody.

In some aspects of the present disclosure, a bispecific antibody or antigen-binding fragment thereof for use in the methods described herein specifically binds to human PD-1 and CTLA-4 and comprises the Heavy Chain (HC) of the MEDI5752 antibody listed in Table 3.

TABLE 3
Full-length heavy chain amino acid sequences

| Anti- | |
|---|---|
| body | Amino Acid Sequence (SEQ ID NO) |
| MEDI | EVQLVESGGGLVQPGGSLRLSCAASGFTFSDYGMHWVRQAP |
| 5752 | GKGLEWVAYISSGSYTIYSADSVKGRFTISRDNAKNSLYLQ |
| PD-1 | MNSLRAEDTAVYYCARRAPNSFYEYYFDYWGQGTTVTVSSA |
| | STKGPSVFPLAPSSKSTSGGTAALGCLVKDYFPEPVTVSWN |
| | SGALTSGVHTFPAVLQSSGLYSLSSVVTVPSSSLGTQTYIC |
| | NVNHKPSNTKVDKRVEPKSCDKTHTCPPCPAPEFEGGPSVF |
| | LFPPKPKDTLMISRTPEVTCVVVDVSHEDPEVKFNWYVDGV |
| | EVHNAKTKPREEQYNSTYRVVSVLTVLHQDWLNGKEYKCKV |
| | SNKALPASIEKTISKAKGQPREPQVCTLPPSREEMTKNQVS |
| | LSCAVKGFYPSDIAVEWESNGQPENNYKTTPPVLDSDGSFF |
| | LVSKLTVDKSRWQQGNVFSCSVMHEALHNHYTQKSLSLSPG |
| | K (SEQ ID NO: 2) |
| MEDI | QVQLVESGGGVVQPGRSLRLSCAASGFTFSSYGMHWVRQAP |
| 5752 | GKGLEWVAVIWYDGSNKYYADSVKGRFTISRDNSKNTLYLQ |
| CTLA- | MNSLRAEDTAVYYCARDPRGATLYYYYYGMDVWGQGTTVTV |
| 4 | SSASTKGPSVCPLAPSSKSTSGGTAALGCLVKDYFPEPVTV |
| | SWNSGALTSGVHTFPAVLQSSGLYSLSSVVTVPSSSLGTQT |
| | YICNVNHKPSNTKVDKRVEPKSVDKTHTCPPCPAPEFEGGP |
| | SVFLFPPKPKDTLMISRTPEVTCVVVDVSHEDPEVKFNWYV |
| | DGVEVHNAKTKPREEQYNSTYRVVSVLTVLHQDWLNGKEYK |
| | CKVSNKALPASIEKTISKAKGQPREPQVYTLPPCREEMTKN |
| | QVSLWCLVKGFYPSDIAVEWESNGQPENNYKTTPPVLDSDG |
| | SFFLYSKLTVDKSRWQQGNVFSCSVMHEALHNHYTQKSLSL |
| | SPGK (SEQ ID NO: 4) |

In some aspects of the present disclosure, a bispecific antibody or antigen-binding fragment thereof for use in the methods described herein specifically binds to human PD-1 and CTL-4 and comprises the Light Chain (LC) of the MEDI5752 antibody listed in Table 4.

TABLE 4
Full-length light chain amino acid sequences

| Anti- | |
|---|---|
| body | Amino Acid Sequence (SEQ ID NO) |
| MEDI | QIVLTQSPATLSLSPGERATLSCSASSKHTNLYWSRHMYWY |
| 5752 | QQKPGQAPRLLIYLTSNRATGIPARFSGSGSGTDFTLTISS |
| PD-1 | LEPEDFAVYYCQQWSSNPFTFGQGTKLEIKRTVAAPSVFIF |
| | PPSDEQLKSGTASVVCLLNNFYPREAKVQWKVDNALQSGNS |
| | QESVTEQDSKDSTYSLSSTLTLSKADYEKHKVYACEVTHQG |
| | LSSPVTKSFNRGEC (SEQ ID NO: 1) |
| MEDI | DIQMTQSPSSLSASVGDRVTITCRASQSINSYLDWYQQKPG |
| 5752 | KAPKLLIYAASSLQSGVPSRFSGSGSGTDFTLTISSLQPED |
| CTLA- | FATYYCQQYYSTPFTFGPGTKVEIKGQPKAAPSVTLFPPCS |
| 4 | EELQANKATLVCLISDFYPGAVTVAWKADSSPVKAGVETTT |
| | PSKQSNNKYAASSYLSLTPEQWKSHRSYSCQVTHEGSTVEK |
| | TVAPTEVS (SEQ ID NO: 3) |

In some aspects, the bispecific antibody or antigen-binding fragment thereof usable in the disclosed methods includes isolated antibodies that bind specifically to human PD-1 and CTLA-4 and cross-compete for binding to human PD-1 and CTLA-4 with MEDI5752. In some aspects, the bispecific antibody or antigen-binding fragment thereof usable in the disclosed methods includes isolated antibodies that bind the same epitope on human PD-1 and CTLA-4 as MEDI5752.

In some aspects, the bispecific antibody or antigen-binding fragment thereof comprises:
(a) a VH CDR1 comprising the amino acid sequence of SEQ ID NO:8, a VH CDR2 comprising the amino acid sequence of SEQ ID NO:9, a VH CDR3 comprising the amino acid sequence of SEQ ID NO:10, a VL CDR1 comprising the amino acid sequence of SEQ ID NO:5, a VL CDR2 comprising the amino acid sequence of SEQ ID NO:6, and a VL CDR3 comprising the amino acid sequence of SEQ ID NO:7; and
(b) a VH CDR1 comprising the amino acid sequence of SEQ ID NO:14, a VH CDR2 comprising the amino acid sequence of SEQ ID NO:15, a VH CDR3 comprising the amino acid sequence of SEQ ID NO:16, a VL CDR1 comprising the amino acid sequence of SEQ ID NO:11, a VL CDR2 comprising the amino acid sequence of SEQ ID NO:12, and a VL CDR3 comprising the amino acid sequence of SEQ ID NO:13.

In some aspects, the bispecific antibody or antigen-binding fragment thereof comprises
(a) a heavy chain comprising the amino acid sequence set forth in SEQ ID NO:2 and a light chain comprising the amino acid sequence set forth in SEQ ID NO:1; and
(b) a heavy chain comprising the amino acid sequence set forth in SEQ ID NO:4 and a light chain comprising the amino acid sequence set forth in SEQ ID NO:3.

In some aspects, the bispecific antibody or antigen-binding fragment comprises an IgG heavy chain constant region. In some aspects, the IgG heavy chain constant region is an IgG1 heavy chain constant region.

In some aspects, the bispecific antibody or antigen-binding fragment thereof is a humanized bispecific antibody or antigen-binding fragment thereof.

As provided herein, an antibody or antigen-binding fragment thereof that immunospecifically binds to PD-1 and CTLA-4 (e.g., human PD-1 and CTLA-4) for use in the methods described herein can have reduced effector function, e.g., as compared to an antibody or antigen-binding fragment thereof with a wild-type IgG1 sequence. The reduced effector function can be, e.g., as a result of the sequence of a constant region of the antibody or antigen-binding fragment thereof.

As provided herein, an antibody or antigen-binding fragment thereof that immunospecifically binds to PD-1 and CTLA-4 (e.g., human PD-1 and CTLA-4) for use in the methods described herein can lack CDC and/or ADCC activity, e.g., as a result of the sequence of the constant region.

### 5.6 Pharmaceutical Compositions

Pharmaceutical compositions suitable for administration to human patients are typically formulated for parenteral administration, e.g., in a liquid carrier, or suitable for reconstitution into liquid solution or suspension for intravenous administration.

In general, such compositions typically comprise a pharmaceutically acceptable carrier. As used herein, the term “pharmaceutically acceptable” means approved by a government regulatory agency or listed in the U.S. Pharmacopeia or another generally recognized pharmacopeia for use in animals, particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil, glycerol polyethylene glycol ricinoleate, and the like. Water or aqueous solution saline and aqueous dextrose and glycerol solutions may be employed as carriers, particularly for injectable solutions. Liquid compositions for parenteral administration can be formulated for administration by injection or continuous infusion. Routes of administration by injection or infusion include intravenous, intraperitoneal, intramuscular, intrathecal and subcutaneous.

The following examples are offered by way of illustration and not by way of limitation.

### 6. EXAMPLES

The examples in this Section (i.e., Section 6) are offered by way of illustration, and not by way of limitation.

### 6.1 Example 1: Phase 1 Clinical Evaluation of MEDI5752

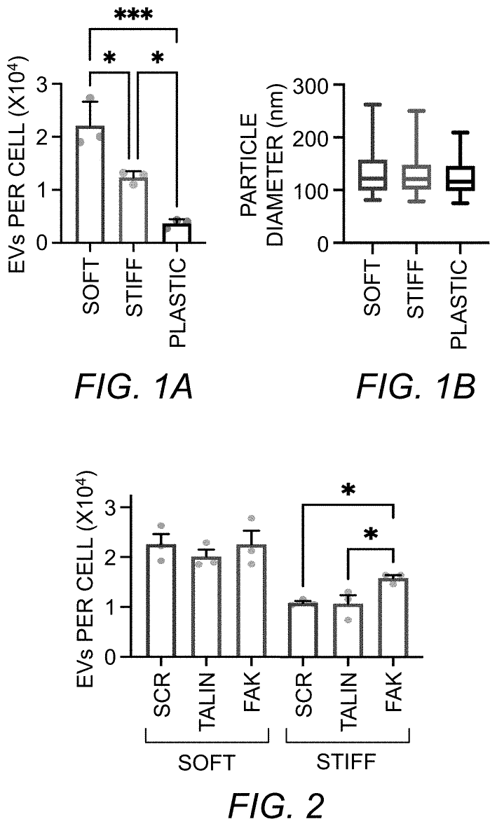

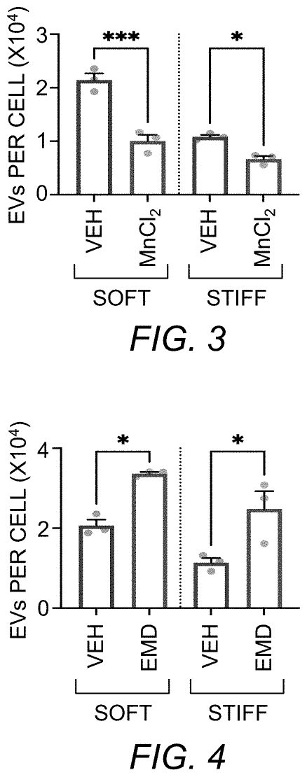

MEDI5752 was assessed in a Phase 1, first-time-in-human, multicenter, open-label, dose-escalation and dose-expansion study to evaluate the safety and tolerability and early evidence of efficacy of MEDI5752 in adult subjects with advanced solid tumors when administered as a single agent or combined with chemotherapy (FIGS. 1A-1D). The purpose of this study was to provide the safety profile, description of pharmacokinetics (PK), pharmacodynamics (PD), and early signs of antitumor efficacy. Up to approximately 396 subjects were to be enrolled in the study: up to approximately 86 subjects in the dose-escalation phase, approximately 92 subjects in the renal cell carcinoma (RCC) expansion cohorts were enrolled, approximately 198 subjects in the non-squamous non-small cell lung cancer (NSCLC) expansion cohorts were enrolled, and approximately 20 subjects in the squamous NSCLC expansion cohort were enrolled.

Subjects were to be enrolled at approximately 50 sites globally. As of the data cut-off date, a total of 178 participants with advanced solid tumors received at least one dose of MEDI5752 monotherapy at one of 10 dose levels (1 each at 2.25 mg and 7.5 mg, 3 at 22.5 mg, 5 at 75 mg, 10 at 225 mg, 20 at 500 mg, 40 at 750 mg, 39 at 1500 mg, 34 at 2000 mg, and 7 at 2500 mg). A total of 152 participants with advanced non-squamous NSCLC received or about to receive at least one dose of MEDI5752 in combination with carboplatin and pemetrexed (˜35 at 500 mg, ˜125 at 750 mg, 20 at 1500 mg, and 7 at 2000 mg). A total of 20 participants with advanced squamous NSCLC received at least one dose of MEDI5752 in combination with carboplatin and paclitaxel or nab-paclitaxel (750 mg). A total of 92 vs 111 participants with renal cell carcinoma received at least one dose of MEDI5752 (33 at 500 mg, 32 at 750 mg, and 27 at 1500 mg).

The study included 2 phases: dose escalation and dose expansion (FIGS. 1A-1D). The dose escalation phase evaluated 10 dose levels to identify a maximum tolerated dose (MTD), optimal biological dose (OBD), or highest protocol-defined dose (HPDD) dose. The dose-escalation phase was followed by the dose-expansion phase, which evaluated 2 cohorts of immunotherapy-naïve subjects with advanced clear-cell RCC (first-line through third-line in RCC-C1 expansion cohort and first-line in RCC-C2 expansion cohort), 2 cohorts of immunotherapy-naïve subjects with first-line Stage IIIB to IV nonsquamous NSCLC (NSCLC expansion cohorts NSCLC-C1 and NSCLC-C2), and 1 cohort of immunotherapy-naïve subjects with first-line Stage IIIB to IV squamous NSCLC (NSCLC expansion cohort NSCLC-C3). Subjects in the RCC-C2 and NSCLC-C1 expansions were randomized to treatments. Subjects remained on treatment until confirmed progressive disease (PD), initiation of alternative cancer therapy, unacceptable toxicity, withdrawal of consent, or other reasons to discontinue treatment occur. All subjects were to be followed for survival until the end of the study (2 years after the final subject had been entered into the study or when the study was stopped, whichever occurred first).

### Dose-Escalation Phase

Dose escalation consisted of 10 dose levels of MEDI5752 (dose levels 2.25, 7.5, 22.5, 75, 225, 500, 750, 1500, 2000, and 2500 mg) administered via IV infusion. Once the OBD, MTD or HPDD was determined, a specific cohort of up to 6 subjects with a glomerular filtration rate (GFR) between 30 and 45 mL/min could be explored at the OBD, MTD, or HPDD (ie, pharmacodynamic cohort). Up to approximately 111 subjects were to be enrolled in the dose-escalation phase.

### Dose-Expansion Phase

The dose-expansion phase was initiated once the MTD, OBD, or HPDD was established in the dose-escalation phase. In the expansion cohorts, only immunotherapy-naïve eligible metastatic subjects were enrolled.

The RCC and NSCLC expansion cohorts provided additional safety, tolerability, efficacy, PK, and PD data for MEDI5752 at several dose levels in these target populations. The NSCLC cohorts also evaluated MEDI5752 in combination with carboplatin plus pemetrexed and carboplatin plus paclitaxel or nab-paclitaxel. In addition, the antitumor activity MEDI5752 in combination with carboplatin plus pemetrexed was compared to that of pembrolizumab in combination with carboplatin plus pemetrexed in subjects with nonsquamous NSCLC.

Subjects in the RCC expansion cohorts (RCC-C1 and RCC-C2) received the following:
MEDI5752 Q3W via IV infusion over 60 minutes (±10 minutes) at 500 mg, 750 mg, and 1500 mg; subjects at 500 mg and 750mg were randomized in a 1:1 ratio when both cohorts were open.
MEDI5752 Q3W via IV infusion over 60 minutes (±10 minutes): a single priming dose at 750 mg, 1000 mg, 1250 mg, or 1500 mg followed by maintenance dose of 225 mg, 500 mg, 750 mg, 1000 mg, or 1250 mg.

Subjects in the nonsquamous NSCLC expansion cohort (NSCLC-C1) received:
Safety lead-in (NSCLC-C1S): MEDI5752 Q3W via IV infusion over 60 minutes (±10 minutes) at a 2000 mg combined with carboplatin area under the concentration-time curve (AUC) 5 mg/mL·min and pemetrexed 500 mg/m2 (Q3W) delivered for 4 doses followed by indefinite maintenance of MEDI5752 and pemetrexed maintenance therapy Q3W
Randomized in a 1:1 ratio (NSCLC-C1R) to receive either:
Arm A: MEDI5752 Q3W via IV infusion over 60 minutes (±10 minutes) at a 1500 mg combined with carboplatin area under the concentration-time curve (AUC) 5 mg/mL·min and pemetrexed 500 mg/m2 (Q3W) delivered for 4 doses followed by indefinite maintenance of MEDI5752 and pemetrexed maintenance therapy Q3W; or
Arm B: Pembrolizumab 200 mg plus carboplatin AUC 5 mg/mL·min and pemetrexed 500 mg/m2 Q3W delivered for 4 doses followed by pembrolizumab for 21 months (a total of 24 months) and indefinite pemetrexed maintenance therapy Q3W.

Subjects in the nonsquamous NSCLC expansion cohort (NSCLC-C2) received
MEDI5752 Q3W via IV infusion over 60 minutes (±10 minutes) at a 500 mg or 750 mg combined with carboplatin area under the concentration-time curve (AUC) 5 mg/mL·min and pemetrexed 500 mg/m2 (Q3W) delivered for 4 doses followed by indefinite maintenance of MEDI5752 and pemetrexed maintenance therapy Q3W

Subjects in the squamous NSCLC expansion cohort (NSCLC-C3) received
MEDI5752 Q3W via IV infusion over 60 minutes (±10 minutes) at a 500 mg or 750 mg combined with carboplatin AUC 6 mg/mL·min Q3W and either paclitaxel 200 mg/m2 Q3W or nab paclitaxel 100 mg/m2 on Days 1, 8, and 15 of each 3-week cycle for 4 doses followed by indefinite MEDI5752 maintenance therapy Q3W

### Endpoints

The primary safety endpoints for the dose-escalation phase were the assessment of the presence of AEs, SAEs, and DLTs and the determination of the MTD, OBD, or HPDD of MEDI5752 in the absence of exceeding the MTD.

The primary objective for the RCC and nonsquamous NSCLC expansion cohorts was to evaluate the preliminary antitumor activity of MEDI5752 monotherapy in RCC and MEDI5752+carboplatin+pemetrexed (versus pembrolizumab+carboplatin+pemetrexed in NSCLC-C1) in nonsquamous NSCLC. The primary endpoint was OR, which is commonly used in oncology studies evaluating subjects with advanced solid tumors, including RCC or NSCLC. The primary objective for the squamous NSCLC expansion cohort was to evaluate the safety and tolerability of MEDI5752 in subjects with advanced solid tumors when combined with chemotherapy.

The secondary endpoints include safety evaluation, additional anti-tumor activity, PK, immunogenicity, and biomarker evaluation.

PD-L1 status will be assessed in subjects who have provided archival or fresh tumor biopsies. For the NSCLC expansion cohorts, PD-L1 positive is defined as baseline PD-L1 expression with tumor cell ≥1% and PD-L1 negative is defined as baseline PD-L1 expression with tumor cell <1%.

### Results

### Safety Data

A total of 136 subjects had been treated with MEDI5752. Table 5 shows the exposure and the MEDI5752-related AEs observed in selected MEDI5752 monotherapy and combination dose levels as of the data snapshot date.

TABLE 5
MEDI5752-related Adverse Events as of 25 Jan. 2023 (Data Snapshot Date)

| | | | | | | Combination with: | | | |
|---|---|---|---|---|---|---|---|---|---|
| | | | | | | Carboplatin and | | | Carboplatin |
| | Monotherapy | | | | | Pemetrexed | | | and Taxane |
| Subjects with | All | 225 mg | 500 mg | 750 mg | 1500 mg | 500 mg | 750 mg | 1500 mg | 750 mg |
| at least one: | N = 178 | N = 10 | N = 38 | N = 40 | N = 39 | N = 33 | N = 72 | N = 20 | N = 18 |
| Median | 3.50 | 3.12 | 5.42 | 3.79 | 3.48 | 2.99 | 4.14 | 4.16 | 2.00 |
| Duration of | | | | | | | | | |
| Exposure | | | | | | | | | |
| (months) | | | | | | | | | |
| Range of | 0.5, 50.4 | 1.3, 50.4 | 0.7, 23.5 | 0.5, 18.6 | 0.5, 14.0 | 0.6, 6.2 | 0.3, 16.4 | 0.7, 25.0 | 0.7, 7.6 |
| Duration of | | | | | | | | | |
| Exposure | | | | | | | | | |
| (months) | | | | | | | | | |
| Median | 4.00 | 4.00 | 7.50 | 5.00 | 4.00 | 4.00 | 5.50 | 4.50 | 3.00 |
| Number of | | | | | | | | | |
| Cycles | | | | | | | | | |
| Range of | 1.0, 71.0 | 2.0, 71.0 | 1.0, 33.0 | 1.0, 27.0 | 1.0, 19.0 | 1.0, 9.0 | 1.0, 17.0 | 1.0, 33.0 | 1.0, 10.0 |
| Cycles | | | | | | | | | |
| AEs (All | 89.3% | 70% | 86.8% | 95% | 94.9% | 72.7% | 80.6% | 100% | 77.8% |
| Grades) | | | | | | | | | |
| Grade 3 or 4 | 46.1% | 10% | 31.6% | 55% | 59% | 18.2% | 36.1% | 70% | 44.4% |
| AEs | | | | | | | | | |
| SAEs | 34.8% | 20% | 18.4% | 45% | 43.6% | 18.2% | 23.6% | 55% | 16.7% |
| AEs Leading to | 38.8% | 0 | 26.3% | 40% | 56.4% | 9.1% | 16.7% | 75% | 11.1% |
| Discontinuation | | | | | | | | | |

AE, adverse event;
N, number;
SAE, serious adverse event

Table 6 provides the exposure and the MEDI5752-related AEs observed in the

RCC cohorts.

TABLE 6
Adverse Events in RCC Cohorts as of 25 Jan. 2023 (Data Snapshot Date)

| | | | Dose | | Dose | | Dose | |
|---|---|---|---|---|---|---|---|---|
| | Dose | | Expansion | | Expansion | | Expansion | |
| MEDI5752 Adverse | Exploration | | 1500 mg | | 750 mg | | 500 mg | |
| Event Profile | (n = 19) | | (n = 27) | | (n = 32) | | (n = 33) | |
| Median duration of | 2.14 | (0.7-15.2) | 3.91 | (0.5-14.0) | 5.52 | (0.7-18.6) | 5.06 | (0.7-13.1) |
| exposure, months (range) | | | | | | | | |
| TEAEs leading to IP | 11 | (57.9) | 20 | (74.1) | 15 | (46.9) | 9 | (27.3) |
| discontinuation, n (%) | | | | | | | | |
| Any grade TRAEs, n (%) | 19 | (100) | 26 | (96.3) | 31 | (96.9) | 29 | (87.9) |
| Grade 3 or 4 TRAEs, n (%) | 13 | (68.4) | 20 | (74.1) | 19 | (59.4) | 10 | (30.3) |

| | | | | | | |
|---|---|---|---|---|---|---|
| Deaths due to TRAE, n (%) | 1 | (5.3)^{‡} | 1 | (3.7)^{†} | 0 | 0 |

TABLE 7
Adverse Events in NSCLC Cohorts as of 25 Jan. 2023 (Data Snapshot Date)

| | Non-squamous | | | | | | | | Squamous | |
|---|---|---|---|---|---|---|---|---|---|---|
| | | | | | Single-arm | | Single-arm | | Single-arm | |
| | Randomised cohort (N = 41) | | | | cohort | | cohort | | cohort | |
| MEDI5752 | MEDI5752 | | | | MEDI5752 | | MEDI5752 | | MEDI5752 | |
| Adverse Event | 1500 mg + CTx | | Pembrolizumab + CTx | | 750 mg + CTx | | 500 mg + CTx | | 750 mg + CTx | |
| Profile | (n = 20) | | (n = 21) | | (n = 72) | | (n = 33) | | (n = 18) | |
| Median duration | 4.5 | (1-33) | NA | | 5.5 | (1-17) | 4.0 | (1-9) | 3.0 | (1-10) |
| of exposure to | | | | | | | | | | |
| MEDI5752, cycles | | | | | | | | | | |
| (range) | | | | | | | | | | |
| Any TEAE, n (%) | 20 | (100.0) | 21 | (100.0) | 69 | (95.8) | 30 | (90.9) | 17 | (94.4) |
| TEAEs leading to | 15 | (75) | 6 | (28.6) | 26 | (36.1) | 4 | (12.1) | 3 | (16.7) |
| IP discontinuation, | | | | | | | | | | |
| n (%) | | | | | | | | | | |
| Any grade TRAEs, | 20 | (100.0) | 21 | (100.0) | 68 | (94.4) | 27 | (81.8) | 17 | (94.4) |
| n (%)* | | | | | | | | | | |
| Grade 3 or 4 | 16 | (80.0) | 13 | (61.9) | 47 | (65.3) | 15 | (45.5) | 13 | (72.2) |

| TRAEs, n (%)* | | | | | | | | | |
|---|---|---|---|---|---|---|---|---|---|
| Deaths due to | 0 | 1 | (4.8)† | 4 | (5.6)‡ | 1 | (3)\hat{ } | 1 | (5.6)\hat{ }\hat{ } |

TRAE, n (%)*

*Related to MEDI5752, pembrolizumab, and/or CTx.
†One patient treated with pembrolizumab + CTx died due to febrile neutropenia.
‡One patient treated with MEDI5752 750 mg + CTx died due to pneumonitis, two due to febrile neutropenia, and one due to septic shock.
\hat{ }One patient treated with MEDI5752 500 mg + CTx died due to febrile neutropenia.
\hat{ }\hat{ }One patient treated with MEDI5752 750 mg + CTx died due to sepsis of respiratory origin.

Dose-limiting toxicities (DLTs) were reported in one subject each in the 2000 mg (Grade 3 pneumonitis and Grade 1 myocarditis in the same subject) and 2500 mg (Grade 3 maculopapular rash) monotherapy cohorts. Two deaths were judged to be related to MEDI5752 treatment; one subject died due to diabetic ketoacidosis and hyperthyroidism and another subject died due to myocardial infarction.

Review of the benefit-risk profile of subjects treated with MEDI5752 suggested that long-term dosing at 1500 mg and above were not suitable for further development due to high treatment discontinuation rates, primarily driven by Grade 3/4 hepatotoxicity.

### Efficacy Data

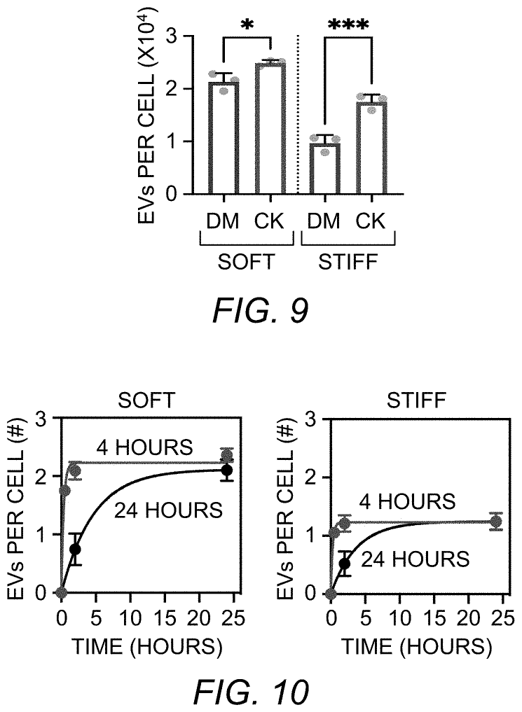

Among 86 MEDI5752 monotherapy subjects in the dose-escalation and maximum tolerated dose (MTD)/optimal biological dose (OBD) cohorts, regardless of dose, in the response evaluable analysis set, the overall objective response rate (ORR) (per Response Evaluation Criteria in Solid Tumors [RECIST] version [v]1.1) was 19.8% (17/86) with 1 complete response (CR). In the 5 subjects treated with 500 mg of MEDI5752 during dose escalation, 2 subjects had a best overall response (B OR) of partial response (PR), 2 subjects had stable disease (SD), and 1 subject had progressive disease (PD). In the 8 subjects treated with 750 mg of MEDI5752 during dose escalation, 4 subjects had a BOR of SD, 3 had PD, and 1 subject was not evaluable. The median duration of response for MEDI5752 monotherapy was 17.5 months (FIG. 9). MEDI5752 monotherapy also showed a durable response in diverse IO-naïve tumors across a range of MEDI5752 doses (FIG. 10).

In the non-small cell lung cancer (NSCLC) expansion cohort (NSCLC-C1R), the ORR in the MEDI5752 1500 mg+carboplatin/pemetrexed arm was 50.0% (10/20) while the ORR in the pembrolizumab+carboplatin/pemetrexed arm was 47.6% (10/21). Table 8 provides the efficacy data in this cohort. See also FIG. 14.

TABLE 8
Efficacy of MEDI5752 1500 mg + carboplatin/pemetrexed arm versus
pembrolizumab + carboplatin/pemetrexed arm in First line Non-
squamous NSCLC as of 25 Jan. 2023 (Data Snapshot Date)

| | MEDI5752 1500 mg | Pembrolizumab |
|---|---|---|
| | + | + |
| | carboplatin/ | carboplatin/ |
| First line Non-squamous | pemetrexed | pemetrexed |
| NSCLC | (n = 20) | (n = 21) |
| Median follow-up, months | 20.1 (0.8-32.5) | 14.5 (1.6-34.3) |
| (range) | | |
| ORR, n (%) | 10 (50.0) | 10 (47.6) |
| Disease control rate, n (%) | 17 (85.0) | 20 (95.2) |
| Median DOR, months (95% | 20.5 (4.1-NE) | 9.9 (2.8-NE) |
| CI) | | |
| Median PFS, months | 15.1 | 8.9 |
| Median OS, months | 25.3 | 16.5 |
| ORR, PD-L1 <1%, n/N (%) | 5/9 (55.6) | 3/10 (30.0) |
| (95% CI) | | |
| Median PFS, PD-L1 <1%, | 13.4 | 9 |
| months | | |

NE, not estimable

In the non-squamous non-small cell lung cancer (NSCLC) expansion cohort (NSCLC-C2), in the first 66 subjects treated with MEDI5752 750 mg+carboplatin/pemetrexed arm, the response-evaluable population, the ORR was 45.5% (30/66). In the PD-L1 <1% subgroup, the ORR was 50% (24/48). The median duration of follow up was 7.2 months (range, 0.3-16.4 months) (FIG. 15B).

In the non-squamous non-small cell lung cancer (NSCLC) expansion cohort (NSCLC-C2), in the subjects treated with MEDI5752 500 mg+carboplatin/pemetrexed arm, the response-evaluable population, the ORR was 25% (8/32), and the preliminary response of CR+PR+unconfirmed PR was 34.4% (11/32). The median duration of follow up was 3.3 months (range, 0.6-6.5 months) (FIG. 15B).

In the squamous non-small cell lung cancer (NSCLC) expansion cohort (NSCLC-C2), in the subjects treated with MEDI5752 750 mg+carboplatin/pemetrexed arm, the response-evaluable population, the ORR was 22.2% (4/18), and the preliminary response of CR+PR+unconfirmed PR was 55.6% (10/18). The median duration of follow up was 2.8 months (range, 0.7-7.6 months) (FIG. 15C).

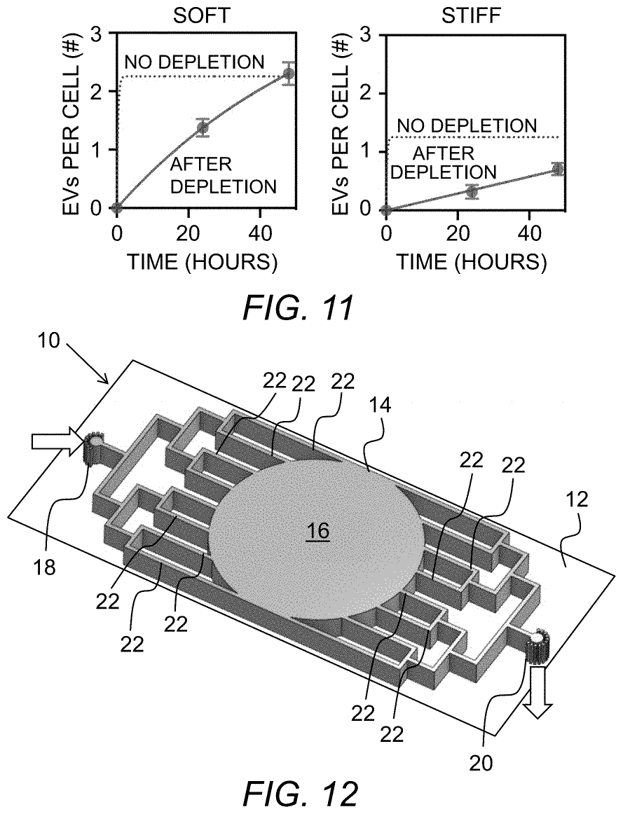

In the renal cell cancer (RCC) expansion cohort receiving MEDI5752 1500 mg (RCC-C1), the ORR was 38.5% (10/26) with 2 CRs. Additionally, the ORR in the first-line RCC subjects was 58.3% (7/12) (FIG. 12). Additionally, the responses for the first-line RCC cohort were found to be durable (FIG. 13). The efficacy of 500 mg and 750 mg is presented in Table 9 (FIG. 12).

TABLE 9
Efficacy of MEDI5752 750 mg and 500 mg in in First line
RCC as of 25 Jan. 2023 (Data Snapshot Date)

| First line Renal Cell Carcinoma | MEDI5752 500 mg | MEDI5752 750 mg |
|---|---|---|
| (with clear cell component) | (n = 33) | (n = 32) |
| Median follow-up, months (range) | 8.6 (1.6-14.7) | 15.9 (2.2-20.4) |
| ORR, n (%) | 15 (45.5) | 15 (46.9) |
| CRs | 2 (6.1) | 3 (9.4) |
| Disease control rate, n (%) | 23 (69.7) | 28 (87.5) |
| ORR, IMDC: Intermediate/poor, | 8/21 (38.1) | 13/24 (54.2) |
| n/N (%) | | |
| CRs | 0 | 2 (8.3) |
| ORR, IMDC: Favorable, n/N (%) | 7/12 (58.3) | 2/8 (25) |
| CRs | 2 (16.7) | 1 (12.5) |

### MEDI5752 Pharmacokinetic/Pharmacodynamic Data

Data suggested that MEDI5752 exhibits nonlinear PK likely due to saturable target-mediated clearance at doses <22.5 mg and additionally likely potentially due to the impact of ADA on clearance of MEDI5752. Mean MEDI5752 PK profiles over the first 84 days are shown in FIG. 4.

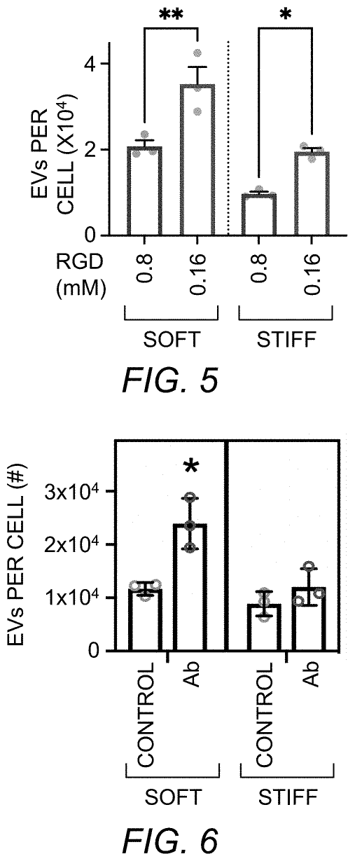

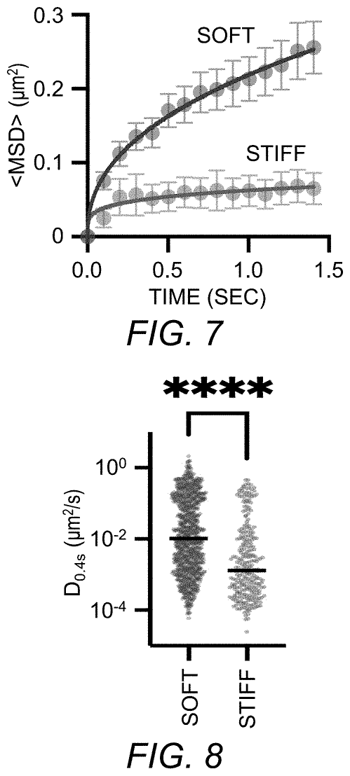

Pharmacodynamic data suggested MEDI5752 leads to a dose-dependent increase in the CD4+ T cell proliferation, plateauing at 500/750 mg and above (FIG. 5 and FIG. 7A). At doses of >225 mg, MEDI5752 demonstrated sustained peripheral PD-1 receptor occupancy (>90%) (FIG. 6). Pharmacodynamic data in monotherapy setting also suggested MEDI5752 leads to a dose-dependent increase in the CD4+ T cell activation, and T cell expansion plateauing at 500/750 mg and above in (FIG. 7B and FIGS. 8A-B respectively). Pharmacodynamic data in chemotherapy combination setting showed a higher increase in the CD4+ T cell proliferation at cycle 1 day 8 and higher proportion of newly expanded T cell clones at cycle 2 at doses of 750 mg and 1500 mg MEDI5752 in combination with chemometherapy in comparaison to pembrolizumab in combination with chemotherapy (FIGS. 7C and 8C respectively).

### 6.2 Example 2: Phase 1b Clinical Evaluation of MEDI5752 in Combination with Axitinib in Subjects with Advanced Renal Cell Carcinoma

MEDI5752 was assessed in a Phase 1b, multicenter, open-label, dose exploration and dose expansion study to evaluate the safety, tolerability, PK, immunogenicity, and antitumor activity of MEDI5752 combined with axitinib and lenvatinib in subjects with advanced RCC not previously treated.

The study included a dose exploration phase followed by a dose expansion phase. The Dose exploration Phase evaluated the safety and tolerability of up to 2 planned dose levels of MEDI5752 in combination with axitinib (Part A) and up to 3-plus additional planned dose levels of MEDI5752 in combination with lenvatinib (Part B). The 2 dose levels of MEDI5752 in combination with axitinib were closed after enrollment of 1 subject each. Therefore, the recommended phase 2 dose (RP2D) was only to be identified for the combination of MEDI5752 with lenvatinib. The RP2D was to be determined by assessing the maximum tolerated dose (MTD) evaluated in the Dose exploration Phase along with the entirety of the clinical data from the rest of the MEDI5752 program. Each dose cohort enrolled up to 9 subjects. Additional subjects could have been required if additional cohorts, treatment schedules, or doses were explored based on emerging PK/pharmacodynamic and clinical data. Once the MTD or MAD of MEDI5752 in combination with lenvatinib was determined in the dose exploration phase, up to 50 subjects were enrolled in the dose expansion phase.

### Target Subject Population

Subjects ≥18 years of age with histologically or cytologically proven advanced RCC with clear cell component that has not been treated previously.

### Treatment Groups and Regimens

Up to approximately 70 subjects were enrolled across 2 phases and allocated to study treatment as detailed below.

### Dose Exploration Phase

The Dose exploration Phase, Part A (MEDI5752+axitinib) included the following 2 planned dose levels:
Dose Level 1 (Starting dose; n=up to 9 subjects) (closed after enrollment of 1 subject):
   Axitinib 5 mg PO BID (Lead-in: Day −7 through Day −1)
   MEDI5752 1500 mg intravenous (IV) every 3 weeks (Q3W) and axitinib 5 mg PO BID (starting on Day 1 [ie, Cycle 1 Day 1])
Dose Level −1 (De-escalation dose; n=up to 9 subjects) (closed after enrollment of 1 subject)
   Axitinib 5 mg PO BID (Lead-in: Day −7 through Day −1)
   MEDI5752 750 mg IV Q3W and axitinib 5 mg PO BID (starting on Day 1 [ie, Cycle 1 Day 1])
      Dose exploration Phase, Part B (MEDI5752+lenvatinib) includes the following 3 planned dose levels:
Dose Level −3 LEN (starting dose; n=up to 9 subjects)
   MEDI5752 500 mg IV Q3W and lenvatinib 14 mg PO QD (starting on Day 1 [ie, Cycle 1 Day 1])
Dose Level −2 LEN (LEN dose escalation; n=up to 9 subjects)
   MEDI5752 500 mg IV Q3W and lenvatinib 18 mg PO QD (starting on Day 1 [ie, Cycle 1 Day 1])
Dose Level −1 LEN (MEDI5752 dose escalation, LEN de-escalation [if necessary]; n=up to 9 subjects)
   MEDI5752 750 mg IV Q3W and lenvatinib (14 mg or 18 mg; dose to be informed by previous two dose levels) PO QD (starting on Day 1 [i.e., Cycle 1 Day 1])
Additional Dose Levels
   MEDI5752 Q3W via IV infusion over 60 minutes (±10 minutes): a single priming dose at 750 mg, 1000 mg, 1250 mg, or 1500 mg followed by maintenance dose of 225 mg, 500 mg, 750 mg, 1000 mg, or 1250 mg and lenvatinib (10 mg, 14 mg, or 18 mg) PO QD (starting on Day 1 of Cycle 1, Cycle 2, or Cycle 3)
   MEDI5752 Q3W via IV infusion over 60 minutes (±10 minutes): at 500 mg or 750 mg and lenvatinib (10 mg) PO QD (starting on Day 1 of Cycle 1)

### Dose-Expansion Phase

MEDI5752 IV Q3W and lenvatinib PO QD (starting on Day 1 [i.e., Cycle 1 Day 1]), administered at the combination dose determined (starting on Day 1 [i.e., Cycle 1 Day 1]) in the Dose exploration Phase (n=up to 50 subjects)

Subjects received MEDI5752 in combination with axitinib or lenvatinib until disease progression, initiation of alternative cancer therapy, unacceptable toxicity, withdrawal of consent, or other reasons to discontinue treatment occur. Subjects were to be followed for survival until the end of study, which was defined as approximately 3 years after the final subject is entered into the study, unless the study closed prior to that time.

### Study Endpoints

Safety: Incidence of AEs/SAEs, dose limiting toxicities, AEs leading to discontinuation of treatment, abnormal laboratory parameters, vital signs, ECG results.

Efficacy: RECIST v1.1 will be used to assess BOR, ORR, DCR, DoR, and TTR in the Response-evaluable population for interim analysis and in the As-treated population for final analysis. PFS (per RECIST v1.1).

Pharmacokinetics: Concentrations of MEDI5752 in serum

Immunogenicity: Incidence of ADAs directed towards MEDI5752.

1.-71. (canceled)

72. A method of treating a renal cell carcinoma (RCC) tumor or a non-small cell lung cancer (NSCLC) tumor in a subject, the method comprising administering to the subject about 250 mg to about 1500 mg of a bispecific antibody or antigen-binding fragment thereof that specifically binds to Programmed cell-death-1 (PD-1) and Cytotoxic T-lymphocyte-associated antigen-4 (CTLA-4).

73. The method of claim 72, comprising administering about 500 mg or about 750 mg of the bispecific antibody or antigen-binding fragment thereof.

74. The method of claim 73, wherein the bispecific antibody is administered once per treatment cycle.

75. The method of claim 74, wherein the treatment cycle is three weeks.

76. The method of claim 72, further comprising (a) administering one or more tyrosine kinase inhibitors, wherein the tyrosine kinase inhibitors are axitinib or levatinib; or (b) administering one or more chemotherapeutic agents.

77. The method of claim 72, wherein the subject is human.

78. The method of claim 72, wherein the bispecific antibody comprises:
(1) (a) a VH CDR1 comprising the amino acid sequence of SEQ ID NO:8, a VH CDR2 comprising the amino acid sequence of SEQ ID NO:9, a VH CDR3 comprising the amino acid sequence of SEQ ID NO:10, a VL CDR1 comprising the amino acid sequence of SEQ ID NO:5, a VL CDR2 comprising the amino acid sequence of SEQ ID NO:6, and a VL CDR3 comprising the amino acid sequence of SEQ ID NO:7; and
(b) a VH CDR1 comprising the amino acid sequence of SEQ ID NO:14, a VH CDR2 comprising the amino acid sequence of SEQ ID NO:15, a VH CDR3 comprising the amino acid sequence of SEQ ID NO:16, a VL CDR1 comprising the amino acid sequence of SEQ ID NO:11, a VL CDR2 comprising the amino acid sequence of SEQ ID NO:12, and a VL CDR3 comprising the amino acid sequence of SEQ ID NO:13; or
(2) (a) a heavy chain comprising the amino acid sequence set forth in SEQ ID NO:2 and a light chain comprising the amino acid sequence set forth in SEQ ID NO:1; and
(b) a heavy chain comprising the amino acid sequence set forth in SEQ ID NO:4 and a light chain comprising the amino acid sequence set forth in SEQ ID NO:3.

79. The method of claim 78, wherein (a) the non-small cell lung cancer and/or the renal cell carcinoma comprises about >50% of the tumor cells expressing PD-L1; (b) the non-small cell lung cancer and/or the renal cell carcinoma comprises about 1-49% of the tumor cells expressing PD-L1; or (c) the non-small cell lung cancer and/or the renal cell carcinoma comprises about <1% of the tumor cells expressing PD-L1.

80. The method of claim 76, wherein the one or more chemotherapeutic agents are selected from the group consisting of carboplatin, pemetrexed, paclitaxel, Nab-paclitaxel, and a combination thereof.

81. A method of expanding T cells in a subject in need thereof, comprising administering a bispecific antibody that specifically binds to Programmed cell-death-1 (PD-1) and Cytotoxic T-lymphocyte-associated antigen-4 (CTLA-4), wherein the bispecific antibody comprises:
(a) a heavy chain comprising the amino acid sequence set forth in SEQ ID NO:2 and a light chain comprising the amino acid sequence set forth in SEQ ID NO:1; and
(b) a heavy chain comprising the amino acid sequence set forth in SEQ ID NO:4 and a light chain comprising the amino acid sequence set forth in SEQ ID NO:3.

82. The method of claim 81, wherein the bispecific antibody is administered at a dose of about 225 mg to about 1,500 mg.

83. The method of claim 82, wherein the bispecific antibody is administered at a dose of about 500 mg or 750 mg once every three weeks.

84. The method of claim 83, wherein the subject has subject has non-small cell lung cancer or renal cell carcinoma.

85. The method of claim 81, wherein the proportion of newly expanded T cell clones is about 50%, about 60%, about 70%, or about 75% higher compared to the number of T cell clones prior to the administration.

86. A method inhibiting growth of a renal cell carcinoma (RCC) tumor or a non-small cell lung cancer (NSCLC) tumor in a subject, the method comprising administering to the subject about 250 mg to about 1500 mg of a bispecific antibody or antigen-binding fragment thereof that specifically binds to Programmed cell-death-1 (PD-1) and Cytotoxic T-lymphocyte-associated antigen-4 (CTLA-4).

87. The method of claim 86, comprising administering about 500 mg or about 750 mg of the bispecific antibody or antigen-binding fragment thereof.

88. The method of claim 87, wherein the bispecific antibody is administered once per treatment cycle.

89. The method of claim 88, wherein the treatment cycle is three weeks.

90. The method of claim 89, wherein the bispecific antibody comprises:
(1) (a) a VH CDR1 comprising the amino acid sequence of SEQ ID NO:8, a VH CDR2 comprising the amino acid sequence of SEQ ID NO:9, a VH CDR3 comprising the amino acid sequence of SEQ ID NO:10, a VL CDR1 comprising the amino acid sequence of SEQ ID NO:5, a VL CDR2 comprising the amino acid sequence of SEQ ID NO:6, and a VL CDR3 comprising the amino acid sequence of SEQ ID NO:7; and
(b) a VH CDR1 comprising the amino acid sequence of SEQ ID NO:14, a VH CDR2 comprising the amino acid sequence of SEQ ID NO:15, a VH CDR3 comprising the amino acid sequence of SEQ ID NO:16, a VL CDR1 comprising the amino acid sequence of SEQ ID NO:11, a VL CDR2 comprising the amino acid sequence of SEQ ID NO:12, and a VL CDR3 comprising the amino acid sequence of SEQ ID NO:13; or
(2) (a) a heavy chain comprising the amino acid sequence set forth in SEQ ID NO:2 and a light chain comprising the amino acid sequence set forth in SEQ ID NO:1; and
(b) a heavy chain comprising the amino acid sequence set forth in SEQ ID NO:4 and a light chain comprising the amino acid sequence set forth in SEQ ID NO:3.
